SEQUENCE LISTING (14 sequences)

SEQ ID NO: 1 - protein, 14 aa, Artificial Sequence
  Synthetic
WLYLDDSGDWWICG

SEQ ID NO: 2 - protein, 14 aa, Artificial Sequence
  Synthetic
WAYLDDSGDWWICG

SEQ ID NO: 3 - protein, 14 aa, Artificial Sequence
  Synthetic
WLALDDSGDWWICG

SEQ ID NO: 4 - protein, 14 aa, Artificial Sequence
  Synthetic
WLYADDSGDWWICG

SEQ ID NO: 5 - protein, 14 aa, Artificial Sequence
  Synthetic
WLYLDDAGDWWICG

SEQ ID NO: 6 - protein, 14 aa, Artificial Sequence
  Synthetic
WLYLDDSADWWICG

SEQ ID NO: 7 - protein, 14 aa, Artificial Sequence
  Synthetic
WLYLDDSGDAWICG

SEQ ID NO: 8 - protein, 14 aa, Artificial Sequence
  Synthetic
WLYLDDSGDWAICG

SEQ ID NO: 9 - protein, 14 aa, Artificial Sequence
  Synthetic
WLYLDDSGDWWACG

SEQ ID NO: 10 - protein, 13 aa, Artificial Sequence
  Synthetic
WLYLDDWDWWICG

SEQ ID NO: 11 - protein, 14 aa, Artificial Sequence
  Synthetic
WLYLDDDWDWWICG

SEQ ID NO: 12 - protein, 14 aa, Artificial Sequence
  Synthetic
WLYLDDYWDWWICG

SEQ ID NO: 13 - protein, 14 aa, Artificial Sequence
  Synthetic
WLYLDDCGDWWICG

SEQ ID NO: 14 - protein, 16 aa, Artificial Sequence
  Synthetic
WLYLDDSGDWWICGAC

(19) United States
(12) Patent Application Publication
(10) Pub. No.: US 2023/0340027 A1
(43) Pub. Date: Oct. 26, 2023

(54) UBIQUITIN HIGH AFFINITY PEPTIDES, AND METHODS OF USING SAME, AND IDENTIFYING SAME

(71) Applicant: TECHNION RESEARCH & DEVELOPMENT FOUNDATION LIMITED, Haifa (IL)

(72) Inventors: Ashraf BRIK, Haifa (IL); Roman MELEDIN, Haifa (IL); Ganga Babu VAMISETTI, Haifa (IL)

(21) Appl. No.: 18/008,515

(22) PCT Filed: Jun. 8, 2021
(86) PCT No.: PCT/IL2021/050686

§ 371 Date: Dec. 6, 2022

Related U.S. Application Data
(60) Provisional application No. 63/035,950, filed on Jun. 8, 2020.

(51) Int. Cl. C07K 7/64

(52) U.S. Cl. CPC C07K 7/64; A61K 38/00

(57) ABSTRACT

The present invention, in some embodiments, is directed to a peptide having increased binding affinity to ubiquitin (Ub). Further provided is a pharmaceutical composition including the peptide of the invention, a method of using same, such as for treating a subject afflicted with cancer, and a method for identifying peptides having increased binding affinity to Ub.

### CROSS-REFERENCE TO RELATED APPLICATIONS

This application claims the benefit of priority to U.S. Provisional Application No. 63/035,950, titled “METHOD FOR SCREENING PEPTIDES”, filed Jun. 8, 2020, the contents of which are incorporated herein by reference in their entirety.

### FIELD OF INVENTION

The present invention, in some embodiments thereof, is in the field of peptide engineering and methods for peptide screening, such as utilizing competitive binding.

### BACKGROUND

Protein-protein interactions (PPIs) regulate numerous essential cellular processes. Exploring those interactions is crucial for understanding cellular events in health and disease and therefore may contribute to drug discovery, as well as fundamental research. Among agents available to target important PPIs macrocyclic peptides (MCPs) have drown special attention. This, due to their advantageous properties of blocking large surfaces of PPIs on the one hand and sufficiently high stability, affinity and target selectivity on the other hand. Thus, MCPs, which lay somewhere between small molecules and protein therapeutics, combine advantages of both drug families.

Development of MCPs with desired properties often involves screening of large libraries, using various approaches. Then the best hits are usually characterized thermodynamically and kinetically using the repertoire of standard biophysical methods, such as Surface Plasmon Resonance (SPR) or Isothermal Titration calorimetry (ITC).

Despite quantitative output of mentioned approaches (e.g. binding affinity) and existence of established protocols, those instruments are not always accessible. Even when available, many of them are unable to perform high-throughput screenings, thus slowing down the process of further product development and optimization.

### SUMMARY

According to a first aspect, there is provided a cyclic peptide comprising the amino acid sequence WLYLDDCGDWWICG (SEQ ID NO: 13).

According to another aspect, there is provided a dimeric cyclic peptide comprising the cyclic peptide of invention.

According to another aspect, there is provided a pharmaceutical composition comprising any one of: (a) the cyclic peptide of the invention; (b) the dimeric cyclic peptide of the invention; and (c) a combination of (a) and (b), and an acceptable carrier.

According to another aspect, there is provided a method for treating a subject afflicted with cancer, the method comprising administering to the subject a therapeutically effective amount of any one of: (a) the cyclic peptide of the invention; (b) the dimeric peptide of the invention; and (c) the pharmaceutical composition of the invention, thereby treating the subject afflicted with cancer.

According to another aspect, there is provided a method for identifying a ubiquitin (Ub) high affinity binding peptide, the method comprising the steps: (a) contacting a poly-Ub protein with a peptide suspected of having increased binding affinity to Ub; and (b) contacting the poly-Ub protein contacted with the suspected peptide of step (a) with a Ub high affinity cyclic peptide comprising a reporter molecule, and determining a signal generated by the reporter molecule, wherein a reduced signal compared to a standard signal is indicative of the suspected peptide has increased binding affinity to the poly-Ub protein, and wherein an increased signal compared to the standard signal is indicative of the suspected peptide has reduced binding affinity to the poly-Ub protein, thereby identifying a Ub high affinity peptide.

In some embodiments, the cysteine residue at position 7 (Cys7) of SEQ ID NO: 13 comprises a chemical modification.

In some embodiments, the chemical modification is selected from the group consisting of: alkylation, arylation and oxidation.

In some embodiments, the peptide comprises not more than 16 amino acids.

In some embodiments, the amino acid at position one of the N terminus is a D amino acid.

In some embodiments, the amino acid at position one of the N terminus is conjugated to a cyclizing molecule.

In some embodiments, the cyclic peptide is prepared using a cyclizing molecule comprising a halogen.

In some embodiments, the cyclizing molecule is selected from the group consisting of: chloracetyl chloride, 3-chlorobenzoyl (3-ClBz), 4-chlorobenzoyl (4-ClBz) or Cl2SAc.

In some embodiments, the dimeric cyclic peptide is a homodimer or a heterodimer.

In some embodiments, the dimeric cyclic peptide comprises two monomeric cyclic peptides covalently linked to one another via cysteine residues.

In some embodiments, the cyclic peptide of the invention or the dimeric cyclic peptide of the invention, is characterized by having: cell penetration capability, ubiquitin (Ub) binding capability, or both.

In some embodiments, the cyclic peptide of the invention or the dimeric cyclic peptide of the invention, is characterized by binding Ub with an affinity Kd ranging from 0.05-100 nM.

In some embodiments, the pharmaceutical composition is characterized by having pro-apoptotic activity.

In some embodiments, the pharmaceutical composition is for use in the treatment of cancer in a subject in need thereof.

In some embodiments, treating comprises reducing deubiquitination activity in at least one cell of the subject.

In some embodiments, treating comprises reducing ubiquitinated proteins proteasomal degradation rate in at least one cell of the subject.

In some embodiments, the suspected peptide is a cyclic peptide.

In some embodiments, the suspected peptide is unlabeled.

In some embodiments, the poly-Ub comprises Ub monomers linked to one another via a Lysine residue.

In some embodiments, the Lysine residue is a Lysine located at position 48 (K48) of the Ub monomers.

In some embodiments, the poly-Ub further comprises a molecule having high binding affinity to a capturing protein.

In some embodiments, the molecule is biotin.

In some embodiments, the capturing protein is streptavidin.

In some embodiments, the capturing protein is immobilized to a surface.

In some embodiments, the method further comprises an additional step comprising removing any one of: (i) suspected peptide unbound to the poly-Ub, (ii) Ub high affinity cyclic peptide unbound to the poly-Ub, and (iii) a combination of (i) and (ii), wherein the additional step is performed between step (b) and step (c).

In some embodiments, the reporter molecule comprises a fluorescent molecule.

In some embodiments, the reporter molecule comprises fluorescein isothiocyanate (FITC).

In some embodiments, the poly-Ub is a tetra Ub poly-Ub.

In some embodiments, the standard signal comprises a signal determined when the poly-Ub is contacted with the Ub high affinity cyclic peptide in the absence of the suspected peptide.

In some embodiments, the identified Ub high affinity peptide is the cyclic peptide of the invention or the dimeric cyclic peptide of the invention.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1G include description of the principles of the herein disclosed competitive tetra-Ub binding assay. (1i) A non-limiting schematic representation of the screening of various unlabeled peptides. (1A) An unlabeled cyclic peptide introduced for binding to the biotin-tetra Ub. (1B) A labeled cyclic peptide is introduced to compete with the binding of the unlabeled peptides to the biotin-tetra Ub followed by washing the unbounded peptides. (1C) The bound peptides are released using Guanidinium-HCl (Gn·HCl) and the fluorescence intensity is measured. (1ii) A non-limiting schematic representation for measuring the dissociation constant (Kd) of FITC-labeled peptide with different concentrations. (1D) A FITC-labeled cyclic peptide with different concentrations are introduced in different wells for binding to the biotin-tetra Ub. (1E) Washing out the unbounded labeled cyclic peptide. (1F) The bound peptides are released using Gn·HCl and the fluorescence intensity is measured. (1G) Binding curve of FITC-labeled Ub4_ix to tetra-Ub K48. The data points were fitted using an equation representing a one site non-cooperative ligand binding model Y=Bmax·X/(Kd+X), where X is ligand (Ub4_ix-FITC) concentration, Y—specific ligand binding, Bmax—maximum specific ligand binding, and Kd—dissociation constant. The Kd value of 35.09±2.66 nM was determined. All measurements were performed in triplicates.

FIGS. 2A-2B include general non-limiting schematic representations of the synthesis of thioether linked cyclic peptides. (2A) Alanine (Ala) scan and mutation at the 7^{th} position. (2B) A non-limiting schematic representation for modification of Cys at the 7^{th} position (Cys7).

FIG. 3 includes a vertical bar graph showing the binding of various thioether linked cyclic peptides to tetra-Ub K48, normalized to the affinity of Ub4_ix. HEPES buffer (Negative control). All measurements were performed in triplicates and in at least three biological replicates. Error bars represent standard error.

FIG. 4 includes graphs and non-limiting schemes describing the synthesis of dimeric cyclic peptides. (a) Dimer formation through disulfide bond (Observed mass: 3567.6±0.4 Da, calculated: 3568.0 Da, average isotopes). (b) Dimer formation through —S—CH₂—S— bridge (Observed mass: 3579.5±0.4 Da, calculated: 3579.9 Da, average isotopes).

FIG. 5 includes graphs and non-limiting schemes describing the synthesis of (Ub4_ix_S7C)₂CH₂—FITC (Observed mass: 4781.6±0.1 Da, calculated: 4782.4 Da, average isotopes). FITC=Fluorescein-5-maleimide.

FIG. 6 includes a vertical bar graph showing the effects of cyclic peptides on cytotoxicity in Hela cells. Cells were exposed for 24 and 48 hours to the indicated (Ub4_ix_S7C)₂CH₂ and Ub4_ix peptide or MG132. Cell viability was measured by FACS (bars represent mean values).

FIG. 7 includes a non-limiting scheme for the measurement of relative binding affinity of peptide through fluoresce based competitive assay.

FIGS. 8A-8C include a scheme, a table and graphs showing the synthesis of different Ub4_ix peptides. (8A) a non-limiting schematic representation for the synthesis of cyclic peptides. (8B) A table of prepared thioether linked cyclic peptides. (8C) HPLC-MS data of each cyclic peptide analogues. ^{a}C4 column was employed for HPLC analysis.

FIGS. 9A-9B includes graphs showing the synthesis of Ub4_ix_S7C cyclic peptide. (9A) HPLC and MS analysis of Ub4_ix_S7C acetamido methyl (Acm) cyclic peptide with observed mass 1854.2±0.1 Da (calculated 1854.3 Da, average isotopes). (9B) HPLC and MS analysis of Ub4_ix_S7C cyclic peptide with observed mass 1783.2±0.1 Da (calculated 1783.4 Da, average isotopes).

FIGS. 10A-10D include graphs showing one-pot synthesis of Ub4_ix_S7C-CH₂CONH₂ cyclic peptide. (10A) HPLC of Ub4_ix_S7C(Acm): peak a corresponds to the peptide with Cys(Acm) with the observed mass 1854.2±0.1 Da (calculated 1855.0 Da, average isotopes). (10B) HPLC of Ub4_ix_S7C: peak b corresponds to the peptide with the free thiol and the observed mass of 1783.2±0.1 Da (calculated 1783.4 Da, average isotopes). (10C) HPLC of the reaction after treatment with 2-iodoacetamide for 2 h: peak c corresponds to the peptide Ub4_ix_S7C-CH₂CONH₂ with observed mass 1840.5±0.1 Da (calculated 1841.0 Da, average isotopes). (10D) HPLC of the reaction after treatment with 2-iodoacetamide for 6 h.

FIG. 11 includes graphs showing HPLC and MS analysis of Ub4_ix_S7C-CH₂C₆H₅ with the observed mass of 1873.3±0.1 Da (calculated 1874.1 Da, average isotopes).

FIG. 12 includes graphs showing HPLC and MS analysis of Ub4_ix_S7C-CH₂C₁₀H₇ cyclic peptide with the observed mass of 1923.5±0.1 Da (calculated 1924.2 Da, average isotopes).

FIG. 13 include graphs showing HPLC and MS analysis of Ub4_ix_S7C-CH₂CH₂NH₂ cyclic peptide with observed mass 1825.9±0.1 Da (calculated 1827.1 Da, average isotopes).

FIG. 14 includes graphs showing HPLC and MS analysis of Ub4_ix_S7C-CH₂C₁₀H₇O₃ cyclic peptide with the observed mass of 1971.5±0.1 Da (calculated 1972.2 Da, average isotopes).

FIG. 15 includes graphs showing HPLC and MS analysis of Ub4_ix_S7C-CH₃cyclic peptide with the observed mass of 1797.2±0.1 Da (calculated 1798.0 Da, average isotopes).

FIG. 16 includes graphs showing HPLC and MS analysis of Ub4_ix_S7C-C₆F₅cyclic peptide with the observed mass of 1949.2±0.1 Da (calculated 1950.0 Da, average isotopes).

FIG. 17 includes graphs showing HPLC and MS analysis of Ub4_ix_S7C(SO₃H) with observed mass 1830.9±0.1 Da (calculated 1831.0 Da, average isotopes).

FIG. 18 includes graphs showing HPLC and mass analysis of dimer with disulfide bond, (Ub4_ix_S7C)₂, cyclic peptide with the observed mass of 3564.6±0.2 Da (calculated 3564.8 Da, average isotopes).

FIG. 19 includes graphs showing HPLC and MS analysis of methylene group stapled dimer, (Ub4_ix_S7C)₂CH₂, cyclic peptide with the observed mass of 3578.5±0.1 Da (calculated 3578.6 Da, average isotopes).

FIG. 20 includes graphs showing HPLC and MS analysis of the cyclic peptide 26 with the observed mass of 2117.4±0.1 Da (calculated 2118.3 Da, average isotopes).

FIG. 21 includes graphs showing HPLC and MS analysis of the cyclic peptide 27 with the observed mass of 2028.4±0.1 Da (calculated 2029.3 Da, average isotopes).

FIG. 22 includes graphs showing HPLC and MS analysis of the cyclic peptide 28 with the observed mass of 2455.4±0.1 Da (calculated 2456.2 Da, average isotopes).

FIG. 23 includes graphs showing HPLC and MS analysis of the cyclic peptide 29 with the observed mass of 2384.4±0.1 Da (calculated 2384.6 Da, average isotopes).

FIG. 24 includes graphs showing HPLC and MS analysis of methylene group stapled dimer with FITC, (Ub4_ix_S7C)₂CH₂-FITC, cyclic peptide with the observed mass of 4781.6±0.1 Da (calculated 4781.7 Da, average isotopes).

FIGS. 25A-25B include a scheme and a graph showing the synthesis of Ub4_ix-FITC cyclic peptide. (25A) A non-limiting schematic representation for the synthesis of Ub4_ix-FITC. (25B) HPLC and MS analysis of FITC labeled cyclic peptide, Ub4_ix-FITC, with the observed mass of 2368.5±0.1 Da (calculated 2368.5 Da, average isotopes).

FIG. 26 includes a graph showing a binding curve of 30, (Ub4_ix_S7C)₂CH₂-FITC to tetra-Ub K48. The K_{d} (12.32±1.26 nM) was determined using formula: Y=Bmax·X/(K_{d}+X). Where Bₘₐₓ=Maximum specific binding, X=Concentration of peptide, Y=Specific binding, K_{d}=dissociation constant. All measurements were performed in triplicates.

FIGS. 27A-27H includes graphs showing the early and late apoptosis of HeLa cells induced by the (Ub4_ix_S7C)₂CH₂. HeLa cells were stained by Annexin V/PI and analyzed by flow cytometry. The figure represents individual dot plots of (27A, 27E) DMSO treated cells. (27B, 27F) Cells treated for 24 or 48 h hours with 0.1 μM of Ub4_ix. (27C, 27G) Cells treated for 24 or 48 h with 0.1 μM of (Ub4_ix_S7C)₂CH₂. (27D, 27H) Cells treated for 24 or 48 h with 0.1 μM of MG132.

FIG. 28 includes micrographs showing the uptake of Ub4_ix-FITC and (Ub4_ix_S7C)₂CH₂-FITC by living cells. HeLa cells were incubated with the cyclic peptides (1 μM) for 2 hours followed by imaging with confocal microscope.

### DETAILED DESCRIPTION

The present invention is directed to a cyclic polypeptide, methods of using same, such as for reducing deubiquitination activity of a cell or for ameliorating, or treating cancer in a subject in need thereof, and methods for identifying same. The present invention is based, in part, on the findings that cyclic peptides bind ubiquitin polymers with an affinity KD at a nanomolar level. The present invention is further based, in part, on the surprising findings that cyclic peptides are capable of penetrating into a cell and increase cell death, e.g., apoptosis.

### Peptides

According to some embodiments, the invention is directed to a peptide.

In some embodiments, the peptide is capable of penetrating a cell (e.g. a cancer cell), binding to ubiquitin, or combination thereof.

In some embodiments, a peptide of the invention comprises or consists of the amino acid sequence: WLYLDDCGDWWICG (SEQ ID NO: 13).

In some embodiments, a peptide of the invention comprises or consists of the amino acid sequence: WAYLDDSGDWWICG (SEQ ID NO: 2).

In some embodiments, a peptide of the invention comprises or consists of the amino acid sequence: WLALDDSGDWWICG (SEQ ID NO: 3).

In some embodiments, a peptide of the invention comprises or consists of the amino acid sequence: WLYADDSGDWWICG (SEQ ID NO: 4).

In some embodiments, a peptide of the invention comprises or consists of the amino acid sequence: WLYLDDAGDWWICG (SEQ ID NO: 5).

In some embodiments, a peptide of the invention comprises or consists of the amino acid sequence: WLYLDDSADWWICG (SEQ ID NO: 6).

In some embodiments, a peptide of the invention comprises or consists of the amino acid sequence: WLYLDDSGDAWICG (SEQ ID NO: 7).

In some embodiments, a peptide of the invention comprises or consists of the amino acid sequence: WLYLDDSGDWAICG (SEQ ID NO: 8).

In some embodiments, a peptide of the invention comprises or consists of the amino acid sequence: WLYLDDSGDWWACG (SEQ ID NO: 9).

In some embodiments, a peptide of the invention comprises or consists of the amino acid sequence: WLYLDDWDWWICG (SEQ ID NO: 10).

In some embodiments, a peptide of the invention comprises or consists of the amino acid sequence: WLYLDDDWDWWICG (SEQ ID NO: 11).

In some embodiments, a peptide of the invention comprises or consists of the amino acid sequence: WLYLDDYWDWWICG (SEQ ID NO: 12).

In some embodiments, the peptide of the invention is linear or cyclic.

As defined herein, the amino acid sequence of a peptide of the invention (SEQ ID Nos.: 1-13) is cited from its N-terminus to the C-terminus. In some embodiments, amino acid residue positioned at the N-terminus of a peptide is located at the first position of the peptide. In some embodiments, a cited position of a given amino acid residue within a cyclic peptide is referred to, based on the position of the amino acid residue in the linear form of the peptide.

In some embodiments, the peptide of the invention has increased affinity to a ubiquitin molecule.

In some embodiments, the cysteine residue at position 7 (Cys7) of SEQ ID NO: 13 comprises a chemical modification or is functionalized.

In some embodiments, the chemical modification is selected from: alkylation, arylation and oxidation.

In some embodiments, functionalized comprises the conjugation of a carbon chain to Cys7 of SEQ ID NO: 13. In some embodiments, a carbon chain comprises one or more carbons. In some embodiments, a carbon chain comprising one or more carbons comprises at least 2, at least 3, at least 4, or at least five carbons, or any value and range therebetween. Each possibility represents a separate embodiment of the invention. In some embodiments, a carbon chain comprising one or more carbons comprises 2-3, 2-4, 2-5, 3-4, 3-5, or 4-5 carbons. Each possibility represents a separate embodiment of the invention. In some embodiments, amino acid residues of a polypeptide of the invention as mentioned above are functionalized by conjugation to a methyl group, ethyl group, propyl group, butyl group, or any combination thereof.

In some embodiments, the peptide comprises not more than 16 amino acids. In some embodiments, not more than 16 amino acid residues comprises 8, 9, 10, 11, 12, 13, 14, or 15 amino acid residues, or any value and range therebetween. Each possibility represents a separate embodiment of the invention. In some embodiments, not more than 16 amino acid residues comprises 7-9, 8-11, 7-12, 10-13, 12-15, 8-14, 9-12, or 10-15 amino acid residues. Each possibility represents a separate embodiment of the invention.

In some embodiments, a peptide of the invention is capable of binding ubiquitin (Ub). In some embodiments, a peptide of the invention has specific binding affinity to ubiquitin (Ub).

As used herein, the term “Ubiquitin” refers to the regulatory protein which is added to other proteins by means of post translational modification. In some embodiments, Ub is a polymeric Ub (poly-Ub). In some embodiments, a polymeric Ub comprises at least 2, at least 3, at least 4, or at least 5 Ub monomers, or any value and range therebetween. Each possibility represents a separate embodiment of the invention. In some embodiments, a polymeric Ub comprises 2-3, 2-4, 2-5, 3-4, 3-5, or 4-5 Ub monomers. Each possibility represents a separate embodiment of the invention. In some embodiments, a Ub polymer comprises Ub monomers linked to one another at lysine (Lys) residue at position 48 (K48). In some embodiments, a Ub polymer comprises Ub monomers linked to one another at Lys residue at position 11 (1(11). In some embodiments, a Ub polymer comprises Ub monomers linked to one another at Lys residue at position 63 (K63). In one embodiment, a Ub polymer comprises Ub monomers linked to one another at K11 and K48. In one embodiment, a Ub polymer comprises Ub monomers linked to one another at K11 and K63. In one embodiment, a Ub polymer comprises Ub monomers linked to one another at K48 and K63. In one embodiment, a Ub polymer comprises Ub monomers linked to one another at K11, K48 and K63. In some embodiments, a polypeptide of the invention has greater binding affinity to poly K48Ub compared to poly K11Ub, or poly K62Ub. In some embodiments, a Ub is further conjugated to a protein. In some embodiments, a protein is post translationally modified by conjugation to a Ub monomer or polymer. In some embodiments, a Ub-conjugated protein is bound by a peptide of the invention at the Ub site. In some embodiments, a peptide of the invention reduces deubiquitination of a Ub-conjugated protein. In some embodiments, a peptide of the invention increases proteasomal degradation of a Ub-conjugated protein.

In some embodiments, a peptide of the invention is capable of binding to Ub in vitro, in vivo, ex vivo, or any combination thereof. In some embodiments, a peptide of the invention is capable of penetrating a cell. In some embodiments, the peptide requires no additional elements to penetrate a cell. In some embodiments, the peptide may be further formulated with other elements for enhancing cell penetration. In some embodiments, the peptide may be used as a carrier or vehicle to carry other elements into a cell.

In some embodiments, the present invention is directed to a cyclic peptide of 12-16 amino acids, capable of penetrating a cell and binding to Ub with affinity KD of 0.1-100 mM.

In some embodiments, a peptide of the invention has increased affinity to Ub compared to control. In some embodiments, increased affinity is by at least 2-fold, at least 3-fold, at least 4-fold, at least 5-fold, at least 6-fold, at least 7-fold, at least 8-fold, at least 9-fold, or at least 10-fold greater compared to control, or any value and range therebetween. Each possibility represents a separate embodiment of the invention. In some embodiments, increased affinity is at least 5%, 10%, 20%, 35%, 50%, 65%, 75%, 85%, 90%, 99%, or 100% greater compared to control, or any value and range therebetween. Each possibility represents a separate embodiment of the invention. In some embodiments, increased affinity is by 1-5%, 4-10%, 8-20%, 25-35%, 30-50%, 45-65%, 60-75%, 70-85%, 80-90%, 85-99%, or 95-100% compared to control. Each possibility represents a separate embodiment of the invention.

As used herein, “control” encompasses any baseline to which the binding affinity of a peptide of the invention to Ub is compared. In some embodiments, a control is a peptide, a polypeptide, or a protein having no Ub binding affinity. In some embodiments, a control is a peptide, a polypeptide, or a protein having low Ub binding affinity. In some embodiments, a control is a peptide, a polypeptide, or a protein known to have Ub binding capabilities, such as ubiquitin receptors and proteins comprising ubiquitin binding domains, non-limiting examples of which include, but are not limited to: Rabex-5, STAM1, STAM2, IsoT, EAP45, S5a/Rpn10, Rpn13, or any other protein known in the art. In some embodiments, a control is a peptide of the invention in its linear form. In some embodiments, a control is a peptide of the invention which is inactivated, such as by antibody neutralization, enzymatic digestion, denaturation, or other methodologies known in the art of protein inactivation, prior to incubation in an environment comprising Ub, in vitro, ex vivo, or in vivo.

In some embodiments, a peptide of the invention has Ub binding affinity with KD of 0.05-1 nM, 0.5-5 nM, 1-10 nM, 5-15 nM, 10-20 nM, 15-30 nM, 20-40 nM, 35-50 nM, 45-60 nM, 55-70 nM, 65-80 nM, 75-90 nM, 85-95 nM, 90-120 nM, 100-500 nM, 250-750 nM, 0.7-1.5 μM, 1-5 μM, 4-10 μM, 8-20 μM, or 15-40 μM. Each possibility represents a separate embodiment of the invention. In some embodiments, a peptide of the invention has Ub binding affinity with KD of 0.1 nM at most, 0.5 nM at most, 1 nM at most, 5 nM at most, 10 nM at most, 20 nM at most, 30 nM at most, 40 nM at most, 50 nM at most, 60 nM at most, 70 nM at most, 80 nM at most, 90 nM at most, 100 nM at most, 110 nM at most, 150 nM at most, 250 nM at most, 500 nM at most, 750 nM at most, 1,500 nM at most, 1 μM at most, 5 μM at most, 10 μM at most, 15 μM at most, 20 μM at most, or 30 μM at most, or any value and range therebetween. Each possibility represents a separate embodiment of the invention.

The present invention encompasses derivatives of the peptide of the invention. The term “derivative” or “chemical derivative” includes any chemical derivative of the peptide having one or more residues chemically derivatized by reaction of side chains or functional groups. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides, which contain one or more naturally occurring amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or serine; and ornithine (O) may be substituted for lysine.

In addition, a peptide derivative can differ from the natural sequence of the peptide of the invention by chemical modifications including, but are not limited to, terminal-NH2 acylation, acetylation, or thioglycolic acid amidation, and by terminal-carboxlyamidation, e.g., with ammonia, methylamine, and the like. Peptides can be either linear, cyclic, or branched and the like, having any conformation, which can be achieved using methods known in the art. In some embodiments, the peptide of the invention further comprises any posttranslational modification (PTM) excluding mammalian naturally occurring PTM.

As used herein, the terms “peptide”, “polypeptide” and “protein” are used herein interchangeably and refer to a polymer of amino acid residues.

The term “amino acid” as used herein means an organic compound containing both a basic amino group and an acidic carboxyl group. Included within this term are naturally occurring amino acids, modified, unusual, non-naturally occurring amino acids, as well as amino acids which are known to occur biologically in free or combined form but usually do not occur in proteins. Included within this term are modified and unusual amino acids, such as those disclosed in, for example, Roberts and Vellaccio (1983) The Peptides. 5: 342-429. Modified, unusual or non-naturally occurring amino acids include, but are not limited to, D-amino acids, hydroxylysine, 4-hydroxyproline, N-Cbz-protected aminovaleric acid (Nva), ornithine (O), aminooctanoic acid (Aoc), 2,4-diaminobutyric acid (Abu), homoarginine, norleucine (Nle), N-methylaminobutyric acid (MeB), 2-naphthylalanine (2Np), aminoheptanoic acid (Ahp), phenylglycine, 1-phenylproline, tert-leucine, 4-aminocyclohexylalanine (Cha), N-methyl-norleucine, 3,4-dehydroproline, N,N-dimethylaminoglycine, N-methylaminoglycine, 4-aminopipetdine-4-carboxylic acid, 6-aminocaproic acid, trans-4-(aminomethyl)-cyclohexanecarboxylic acid, 2-, 3-, and 4-(aminomethyl)-benzoic acid, 1-aminocyclopentanecarboxylic acid, 1-aminocyclopropanecarboxylic acid, cyano-propionic acid, 2-benzyl-5-aminopentanoic acid, Norvaline (Nva), 4-O-methyl-threonine (TMe), 5-O-methyl-homoserine (hSM), tert-butyl-alanine (tBu), cyclopentyl-alanine (Cpa), 2-amino-isobutyric acid (Aib), N-methyl-glycine (MeG), N-methyl-alanine (MeA), N-methyl-phenylalanine (MeF), 2-thienyl-alanine (2Th), 3-thienyl-alanine (3Th), O-methyl-tyrosine (YMe), 3-Benzothienyl-alanine (Bzt) and D-alanine (DAl).

The term “amino acid residue” as used herein refers to the portion of an amino acid that is present in a peptide.

The term “peptide bond” means a covalent amide linkage formed by loss of a molecule of water between the carboxyl group of one ammo acid and the ammo group of a second ammo acid.

The terms “peptide”, “polypeptide” and “protein” as used herein encompass native peptides, peptidomimetics (typically including non-peptide bonds or other synthetic modifications) and the peptide analogs peptoids and semi-peptoids or any combination thereof. In another embodiment, the terms “peptide”, “polypeptide” and “protein” apply to amino acid polymers in which at least one amino acid residue is an artificial chemical analog of a corresponding naturally occurring amino acid.

One of skill in the art will recognize that individual substitutions, deletions or additions to a peptide, or protein sequence which alters, adds or deletes a single amino acid or a small percentage of amino acids in the encoded sequence is a conservatively modified variant where the alteration results in the substitution of an amino acid with a similar charge, size, and/or hydrophobicity characteristics, such as, for example, substitution of a glutamic acid (E) to an aspartic acid (D).

As used herein, the phrase “conservative substitution” also includes the use of a chemically derivatized residue in place of a non-derivatized residue provided that such peptide displays the requisite function as specified herein.

The peptide derivatives according to the principles of the present invention can also include side chain bond modifications, including but not limited to —CH2-NH—, —CH2-S—, —CH2-S=0, OC—NH—, —CH2-O—, —CH2-CH2-, S═C—NH—, and —CH═CH—, and backbone modifications such as modified peptide bonds. Peptide bonds (—CO—NH—) within the peptide can be substituted, for example, by N-methylated bonds (—N(CH3)-CO—); ester bonds (—C(R)H—C—O—O—C(R)H—N); ketomethylene bonds (—CO—CH2-); a-aza bonds (—NH—N(R)—CO—), wherein R is any alkyl group, e.g., methyl; carba bonds (—CH2-NH—); hydroxyethylene bonds (—CH(OH)—CH2-); thioamide bonds (—CS—NH); olefmic double bonds (—CH═CH—); and peptide derivatives (—N(R)—CH2-CO—), wherein R is the “normal” side chain, naturally presented on the carbon atom. These modifications can occur at one or more of the bonds along the peptide chain and even at several (e.g., 2-3) at the same time.

The invention further includes peptides and derivatives thereof, which can contain one or more D-isomer forms of the amino acids. Production of retro-inverso D-amino acid peptides where at least one amino acid and perhaps all amino acids are D-amino acids is well known in the art. When all of the amino acids in the peptide are D-amino acids, and the N- and C-terminals of the molecule are reversed, the result is a molecule having the same structural groups being at the same positions as in the L-amino acid form of the molecule. However, the molecule is more stable to proteolytic degradation and is therefore useful in many of the applications recited herein. Diastereomeric peptides may be highly advantageous over all L- or all D-amino acid peptides having the same amino acid sequence because of their higher water solubility, lower immunogenicity, and lower susceptibility to proteolytic degradation. The term “diastereomeric peptide” as used herein refers to a peptide comprising both L-amino acid residues and D-amino acid residues. The number and position of D-amino acid residues in a diastereomeric peptide of the preset invention may be variable so long as the peptide is capable of displaying the function of disclosed chimera of the invention.

In some embodiments, the amino acid at position one of the N terminus is a D amino acid.

In some embodiments, the peptide of the invention is cyclized. In some embodiments, the first amino acid residue of the N terminus of a peptide of the invention is conjugated to a cyclizing molecule. In some embodiments, the first amino acid residue of the peptide (positioned at the N terminus) and another amino residue of the peptide (positioned at the C terminus) are bound to one another, thereby resulting in a cyclic peptide. In some embodiments, the cyclizing molecule is bound to both the first amino acid residue and to another amino acid residue located at the C terminus. In some embodiments, the cyclizing molecule facilitates the binding of the first and the C terminal amino acid residue. In some embodiments, the cyclizing molecule is released upon binding of the first and the C terminal amino acid residue. In some embodiments, the cyclizing molecule is conjugated to both the first and the C terminal amino acid residue upon binding. As defined herein, a “C terminal amino acid residue” refers to an amino acid residue located in a position closer to the C terminal end of a linear peptide compared to the N terminal end of the peptide. In some embodiments, a C terminal amino acid residue is positioned 8 before last, 7 before last, 6 before last, 5 before last, 4 before last, 3 before last, 2 before last, 1 before last, or is the last amino acid residue in a linear peptide. Each possibility represents a separate embodiment of the invention. According to a non-limiting example, an amino acid residue at position 9 of a peptide comprising 16 amino acid residues is considered as a C terminal amino acid residue. A variety of methods are available for cyclizing a polypeptide (e.g., macrocyclization) as reviewed, for example by White and Yudin (2011).

In some embodiments, a cyclizing molecule comprises one or more halogen atoms selected from: Fluoride (F), Chlorine (Cl), Bromide (Br), Iodine (I) and Astatine (At), or any combination thereof. Non-limiting examples for a cyclizing molecule comprising a halogen include, but are not limited to: chloracetyl chloride, 3-chlorobenzoyl (3-ClBz), 4-chlorobenzoyl (4-ClBz) or C12SAc. In one embodiment, a cyclizing molecule comprising a halogen group is conjugated to the first amino acid residue of a polypeptide's N terminus and nucleophilically attacks a thiol group of a cysteine residue located at the C terminal end of the peptide, thereby resulting in a cyclic peptide.

In some embodiments, the cyclizing molecule is selected from: chloracetyl chloride, 3-chlorobenzoyl (3-ClBz), 4-chlorobenzoyl (4-ClBz) or C12SAc.

In some embodiments, the peptide is prepared using a cyclizing molecule comprising a halogen.

In some embodiments, there is provided a dimeric peptide comprising the peptide of the invention.

In some embodiments, there is provided a dimeric cyclic peptide comprising the cyclic peptide of the invention.

In some embodiments, the dimeric peptide is a homodimer or a heterodimer. In some embodiments, the dimeric peptide is a homodimer.

In some embodiments, the dimeric peptide comprises two monomeric peptides covalently linked to one another via cysteine residues.

In some embodiments, the monomeric peptides are linked via a disulfide bond (—S—S—). In some embodiments, the monomeric peptides are linked via a linker, e.g., a carbon chain of one or more carbon atoms. In some embodiments, the monomeric peptides are linked via a CH₂ linker. In some embodiments, the monomeric peptides are linked via the following bond: —S—CH2-S—.

In some embodiments, any one of the peptide of the invention and the dimeric peptide of the invention, is characterized by having: cell penetration capability, ubiquitin (Ub) binding capability, or both.

In some embodiments, any one of the peptide of the invention and the dimeric peptide of the invention, is characterized by binding Ub with an affinity K_{d} ranging from 0.05-100 nM.

### Polypeptide Synthesis

According to one embodiment, the peptide of the invention may be synthesized or prepared by any method and/or technique known in the art for peptide synthesis. According to another embodiment, the polypeptide may be synthesized by a solid phase peptide synthesis method of Merrifield (see J. Am. Chem. Soc, 85:2149, 1964). According to another embodiment, the peptide of the invention can be synthesized using standard solution methods, which are well known in the art (see, for example, Bodanszky, M., Principles of Peptide Synthesis, Springer-Verlag, 1984).

In general, the synthesis methods comprise sequential addition of one or more amino acids or suitably protected amino acids to a growing peptide chain bound to a suitable resin. Normally, either the amino or carboxyl group of the first amino acid is protected by a suitable protecting group. The protected or derivatized amino acid can then be either attached to an inert solid support (resin) or utilized in solution by adding the next amino acid in the sequence having the complimentary (amino or carboxyl) group suitably protected, under conditions conductive for forming the amide linkage. The protecting group is then removed from this newly added amino acid residue and the next amino acid (suitably protected) is added, and so forth. After all the desired amino acids have been linked in the proper sequence, any remaining protecting groups are removed sequentially or concurrently, and the peptide chain, if synthesized by the solid phase method, is cleaved from the solid support to afford the final peptide.

In the solid phase peptide synthesis method, the alpha-amino group of the amino acid is protected by an acid or base sensitive group. Such protecting groups should have the properties of being stable to the conditions of peptide linkage formation, while being readily removable without destruction of the growing peptide chain. Suitable protecting groups are t-butyloxycarbonyl (BOC), benzyloxycarbonyl (Cbz), biphenylisopropyloxycarbonyl, t-amyloxycarbonyl, isobornyloxycarbonyl, (alpha, alpha)-dimethyl-3,5 dimethoxybenzyloxycarbonyl, o-nitrophenyl sulfenyl, 2-cyano-t-butyloxycarbonyl, 9-fluorenylmethyloxycarbonyl (Fmoc) and the like. In the solid phase peptide synthesis method, the C-terminal amino acid is attached to a suitable solid support. Suitable solid supports useful for the above synthesis are those materials, which are inert to the reagents and reaction conditions of the stepwise condensation-deprotection reactions, as well as being insoluble in the solvent media used. Suitable solid supports are chloromethylpolystyrene-divinylbenzene polymer, hydroxymethyl-polystyrene-divinylbenzene polymer, and the like. The coupling reaction is accomplished in a solvent such as ethanol, acetonitrile, N,N-dimethylformamide (DMF), and the like. The coupling of successive protected amino acids can be carried out in an automatic polypeptide synthesizer as is well known in the art.

In another embodiment, a peptide of the invention may be synthesized such that one or more of the bonds, which link the amino acid residues of the peptide are non-peptide bonds. In another embodiment, the non-peptide bonds include, but are not limited to, imino, ester, hydrazide, semicarbazide, and azo bonds, which can be formed by reactions well known to one skilled in the art.

The term “linker” refers to a molecule or macromolecule serving to connect different moieties of a peptide or a polypeptide. In one embodiment, a linker may also facilitate other functions, including, but not limited to, preserving biological activity, maintaining sub-units and domains interactions, and others.

In some embodiments, a peptide of the invention may be attached or linked to another molecule via a chemical linker. Chemical linkers are well known in the art and include, but are not limited to, dicyclohexylcarbodiimide (DCC), N-hydroxysuccinimide (NHS), maleiimidobenzoyl-N-hydroxysuccinimide ester (MBS), N-ethyloxycarbonyl-2-ethyloxy-1,2-dihydroquinoline (EEDQ), N-isobutyloxy-carbonyl-2-isobutyloxy-1,2-dihydroquinoline (IIDQ). In another embodiment, linkers may also be monomeric entities such as a single amino acid. In another embodiment, amino acids with small side chains are especially preferred, or a small polypeptide chain, or polymeric entities of several amino acids. In another embodiment, a polypeptide linker is fifteen amino acids long or less, ten amino acids long or less, or five amino acids long or less. In one embodiment, a linker may be a nucleic acid encoding a small polypeptide chain. In another embodiment, a linker encodes a polypeptide linker of fifteen amino acids long or less, ten amino acids long or less, or five amino acids long or less.

Recombinant technology may be used to express the peptide of the invention, and is well known in the art. In another embodiment, the linker may be a cleavable linker, resulting in cleavage of the peptide of the invention once delivered to the tissue or cell of choice. In such an embodiment, the cell or tissue would have endogenous (either naturally occurring enzyme or be recombinantly engineered to express the enzyme) or have exogenous (e.g., by injection, absorption or the like) enzyme capable of cleaving the cleavable linker.

In another embodiment, the linker may be biodegradable such that the polypeptide of the invention is further processed by hydrolysis and/or enzymatic cleavage inside cells. In one embodiment, tumor specifically-expressed proteases, can be used in the delivery of prodrugs of cytotoxic agents, with the linker being selective for a site-specific proteolysis. In some embodiments, a readily-cleavable group include acetyl, trimethylacetyl, butanoyl, methyl succinoyl, t-butyl succinoyl, ethoxycarbonyl, methoxycarbonyl, benzoyl, 3-aminocyclohexylidenyl, and the like.

The invention further encompasses a polynucleotide sequence comprising a nucleic acid encoding any of the peptides of the invention. In another embodiment, the nucleic acid sequence encoding the peptide is at least 70%, or alternatively at least 80%, or alternatively at least 90%, or alternatively at least 95%, or alternatively at least 99% homologous to the nucleic acid sequence encoding the nucleic acid sequence of the peptides of the invention or a derivative thereof, or any value and range therebetween. Each possibility represents a separate embodiment of the invention.

In some embodiments, the invention provides a polynucleotide encoding the peptide of the invention.

In some embodiments, a polynucleotide molecule of the invention encodes a peptide comprising non-canonical amino acids.

In some embodiments, the polynucleotide of the invention is ligated into an expression vector, comprising a transcriptional control of a cis-regulatory sequence (e.g., promoter sequence). In some embodiments, the cis-regulatory sequence is suitable for directing constitutive expression of the polypeptide of the invention. In some embodiments, the cis-regulatory sequence is suitable for directing tissue-specific expression of the polypeptide of the invention. In some embodiments, the cis-regulatory sequence is suitable for directing inducible expression of the polypeptide of the invention.

The term “polynucleotide” refers to a nucleic acid (e.g., DNA or RNA) sequence that comprises coding sequences necessary for the production of a polypeptide. In one embodiment, a polynucleotide refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

In one embodiment, “complementary polynucleotide sequence” refers to a sequence, which results from reverse transcription of messenger RNA using a reverse transcriptase or any other RNA-dependent DNA polymerase. In one embodiment, the sequence can be subsequently amplified in vivo or in vitro using a DNA polymerase.

In one embodiment, “genomic polynucleotide sequence” refers to a sequence derived (or isolated) from a chromosome and, thus it represents a contiguous portion of a chromosome.

In one embodiment, “composite polynucleotide sequence” refers to a sequence, which is at least partially complementary and at least partially genomic. In one embodiment, a composite sequence can include some exonal sequences required to encode the polypeptide of the invention, as well as some intronic sequences interposing therebetween. In one embodiment, the intronic sequences can be of any source, including of other genes, and typically may include conserved splicing signal sequences. In one embodiment, intronic sequences include cis-acting expression regulatory elements.

In some embodiments, a polynucleotide of the invention is prepared using PCR techniques, or any other method or procedure known to one of ordinary skill in the art.

In one embodiment, a polynucleotide of the invention is inserted into expression vectors (i.e., a nucleic acid construct) to enable expression of a recombinant polypeptide. In one embodiment, the expression vector includes additional sequences which render this vector suitable for replication and integration in prokaryotes. In one embodiment, the expression vector includes additional sequences which render this vector suitable for replication and integration in eukaryotes. In one embodiment, the expression vector includes a shuttle vector which renders this vector suitable for replication and integration in both prokaryotes and eukaryotes. In some embodiments, cloning vectors comprise transcription and translation initiation sequences (e.g., promoters, enhancers) and transcription and translation terminators (e.g., polyadenylation signals).

In one embodiment, a variety of prokaryotic or eukaryotic cells can be used as host-expression systems to express the polypeptide of the invention. In some embodiments, these include, but are not limited to, microorganisms, such as bacteria transformed with a recombinant bacteriophage DNA, plasmid DNA or cosmid DNA expression vector containing the polypeptide coding sequence; yeast transformed with recombinant yeast expression vectors containing the polypeptide coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the polypeptide coding sequence.

In some embodiments, non-bacterial expression systems are used (e.g. mammalian expression systems) to express the polypeptide of the invention. In one embodiment, the expression vector is used to express the polynucleotide of the invention in mammalian cells.

In some embodiments, in bacterial systems, a number of expression vectors can be advantageously selected depending upon the use intended for the polypeptide expressed. In one embodiment, large quantities of polypeptide are desired. In one embodiment, vectors that direct the expression of high levels of the protein product, possibly as a fusion with a hydrophobic signal sequence, which directs the expressed product into the periplasm of the bacteria or the culture medium where the protein product is readily purified are desired. In one embodiment, certain fusion protein engineered with a specific cleavage site to aid in recovery of the polypeptide. In one embodiment, vectors adaptable to such manipulation include, but are not limited to, the pET series of E. coli expression vectors [Studier et al., Methods in Enzymol. 185:60-89 (1990)].

In one embodiment, yeast expression systems are used. In one embodiment, a number of vectors containing constitutive or inducible promoters can be used in yeast as disclosed in U.S. Pat. No. 5,932,447. In another embodiment, vectors which promote integration of foreign DNA sequences into the yeast chromosome are used.

In one embodiment, the expression vector may further include additional polynucleotide sequences that allow, for example, the translation of several proteins from a single mRNA such as an internal ribosome entry site (IRES).

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses can be used. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression of the polypeptide of the invention. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, the viral vectors that are produced are unable to spread laterally. In one embodiment, this characteristic can be useful if the desired purpose is to introduce a specified gene into only a localized number of targeted cells.

Various methods can be used to introduce an expression vector into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In one embodiment, plant expression vectors are used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct can also include sequences engineered to optimize stability, production, purification, yield or activity of the expressed polypeptide.

In some embodiments, transformed cells are cultured under effective conditions, which allow for the expression of high amounts of a recombinant polypeptide. In some embodiments, effective culture conditions include, but are not limited to, effective media, bioreactor, temperature, pH and oxygen conditions that permit protein production. In one embodiment, an effective medium refers to any medium in which a cell is cultured to produce a recombinant polypeptide of the present invention. In some embodiments, a medium typically includes an aqueous solution having assimilable carbon, nitrogen and phosphate sources, and appropriate salts, minerals, metals and other nutrients, such as vitamins. In some embodiments, the cells can be cultured in conventional fermentation bioreactors, shake flasks, test tubes, microtiter dishes and petri plates. In some embodiments, culturing is carried out at a temperature, pH and oxygen content appropriate for a recombinant cell. In some embodiments, culturing conditions are within the expertise of one of ordinary skill in the art.

In some embodiments, depending on the vector and host system used for production, resultant polypeptide of the invention either remains within the recombinant cell, secreted into the fermentation medium, secreted into a space between two cellular membranes, such as the periplasmic space in E. coli; or retained on the outer surface of a cell or viral membrane. In one embodiment, following a predetermined time in culture, recovery of the recombinant polypeptide is affected.

In one embodiment, the phrase “recovering the recombinant polypeptide” as used herein, refers to collecting the whole fermentation medium containing the peptide and need not imply additional steps of separation or purification.

In one embodiment, a peptide of the invention is purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

In one embodiment, to facilitate recovery, the expressed coding sequence can be engineered to encode the polypeptide of the invention and fused cleavable moiety. In one embodiment, a fusion protein can be designed so that the polypeptide can be readily isolated by affinity chromatography; e.g., by immobilization on a column specific for the cleavable moiety. In one embodiment, a cleavage site is engineered between the polypeptide and the cleavable moiety, and the polypeptide can be released from the chromatographic column by treatment with an appropriate enzyme or agent that specifically cleaves the fusion protein at this site [e.g., see Booth et al., Immunol. Lett. 19:65-70 (1988); and Gardella et al., J. Biol. Chem. 265:15854-15859 (1990)].

In one embodiment, the peptide of the invention is retrieved in “substantially pure” form that allows for the effective use of the protein in the applications described herein.

As used herein, the term “substantially pure” describes a peptide/polypeptide or other material which has been separated from its native contaminants. Typically, a monomeric peptide is substantially pure when at least about 60 to 75% of a sample exhibits a single peptide backbone. Minor variants or chemical modifications typically share the same peptide sequence. A substantially pure peptide can comprise over about 85 to 90% of a peptide sample, and can be over 95% pure, over 97% pure, or over about 99% pure, or any value and range therebetween. Each possibility represents a separate embodiment of the invention. Purity can be measured on a polyacrylamide gel, with homogeneity determined by staining. Alternatively, for certain purposes high resolution may be necessary and HPLC or a similar means for purification can be used. For most purposes, a simple chromatography column or polyacrylamide gel can be used to determine purity.

The term “purified” does not require the material to be present in a form exhibiting absolute purity, exclusive of the presence of other compounds. Rather, it is a relative definition. A peptide is in the “purified” state after purification of the starting material or of the natural material by at least one order of magnitude, 2 or 3, or 4 or 5 orders of magnitude.

In one embodiment, the peptide of the invention is substantially free of naturally-associated host cell components. The term “substantially free of naturally-associated host cell components” describes a peptide or other material which is separated from the native contaminants which accompany it in its natural host cell state. Thus, a peptide which is chemically synthesized or synthesized in a cellular system different from the host cell from which it naturally originates will be free from its naturally-associated host cell components.

In one embodiment, the peptide of the invention can also be synthesized using in vitro expression systems. In one embodiment, in vitro synthesis methods are well known in the art and the components of the system are commercially available. Non-limited example for in vitro system includes but is not limited to in vitro translation.

### Pharmaceutical Composition

According to some embodiments, there is provided a pharmaceutical composition comprising any one of: (a) the peptide of the invention; (b) the dimeric peptide of the invention; and (c) a combination of (a) and (b), and an acceptable carrier.

In some embodiments, the pharmaceutical composition is characterized by having pro-apoptotic activity.

As used herein, the term “pro-apoptotic activity” refers to a compound's ability to induce, increase, enhance, propagate, facilitate, contribute, being involved, or any combination thereof, with programed cell death.

In some embodiments, the pharmaceutical composition is for use in the treatment of cancer in a subject in need thereof.

In some embodiments, the pharmaceutical composition facilitates administration of a compound to an organism. According to another embodiment, the invention provides a pharmaceutical composition comprising as an active ingredient a therapeutically effective amount of the peptide of the invention, the dimeric peptide of the invention, or both.

In another embodiment, the pharmaceutical composition of the invention may be formulated in the form of a pharmaceutically acceptable salt of the peptides of the present invention or their analogs, or derivatives thereof. In another embodiment, pharmaceutically acceptable salts include those salts formed with free amino groups such as salts derived from non-toxic inorganic or organic acids such as hydrochloric, phosphoric, acetic, oxalic, tartaric acids, and the like, and those salts formed with free carboxyl groups such as salts derived from non-toxic inorganic or organic bases such as sodium, potassium, ammonium, calcium, ferric hydroxides, isopropylamine, triethylamine, 2-ethylamino ethanol, histidine, procaine, and the like.

As used herein, the term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; and agents for the adjustment of tonicity such as sodium chloride or dextrose are also envisioned. The carrier may comprise, in total, from about 0.1% to about 99.99999% by weight of the pharmaceutical compositions presented herein.

As used herein, the term “pharmaceutically acceptable” means suitable for administration to a subject, e.g., a human. For example, the term “pharmaceutically acceptable” can mean approved by a regulatory agency of the Federal or a state government or listed in the U. S. Pharmacopeia or other generally recognized pharmacopeia for use in animals, and more particularly in humans.

In another embodiment, the compositions of the invention take the form of solutions, suspensions, emulsions, tablets, pills, capsules, powders, gels, creams, ointments, foams, pastes, sustained-release formulations and the like. In another embodiment, the compositions of the invention can be formulated as a suppository, with traditional binders and carriers such as triglycerides, microcrystalline cellulose, gum tragacanth or gelatin. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in: Remington's Pharmaceutical Sciences” by E. W. Martin, the contents of which are hereby incorporated by reference herein. Such compositions will contain a therapeutically effective amount of the polypeptide of the invention, preferably in a substantially purified form, together with a suitable amount of carrier so as to provide the form for proper administration to the subject.

According to an embodiment of the invention, pharmaceutical compositions contain 0.1-95% of the polypeptide(s) of the invention, derivatives, or analogs thereof. According to another embodiment of the invention, pharmaceutical compositions contain 1-70% of the polypeptide(s). According to another embodiment of the invention, the composition or formulation to be administered may contain a quantity of polypeptide(s), according to embodiments of the invention in an amount effective to treat the condition or disease of the subject being treated.

An embodiment of the invention relates to a peptide, dimeric peptide, or both, of the invention, presented in unit dosage form and prepared by any of the methods well known in the art of pharmacy. In an embodiment of the invention, the unit dosage form is in the form of a tablet, capsule, lozenge, wafer, patch, ampoule, vial or pre-filled syringe. In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems.

According to one embodiment, the compositions of the invention are administered in the form of a pharmaceutical composition comprising at least one of the active components of this invention (e.g., cyclic peptide) together with a pharmaceutically acceptable carrier or diluent. In another embodiment, the compositions of this invention can be administered either individually or together in any conventional oral, parenteral or transdermal dosage form. In some embodiments, the pharmaceutical composition further comprises at least one anticancer agent such as a chemotherapeutic agent. In some embodiments, the pharmaceutical composition is adopted for combined administration with an anticancer therapy such as chemotherapy, radiotherapy, immunotherapy, hormonal therapy, toxin therapy or surgery.

As used herein, the terms “administering”, “administration”, and like terms refer to any method which, in sound medical practice, delivers a composition containing an active agent to a subject in such a manner as to provide a therapeutic effect.

Depending on the location of the tissue of interest, the peptide of the invention can be administered in any manner suitable for the provision of the peptide to cells within the tissue of interest. Thus, for example, a composition containing the peptide of the invention can be introduced, for example, into the systemic circulation, which will distribute the peptide to the tissue of interest. Alternatively, a composition can be applied topically to the tissue of interest (e.g., injected, or pumped as a continuous infusion, or as a bolus within a tissue, applied to all or a portion of the surface of the skin, etc.).

In some embodiments, the pharmaceutical compositions comprising the peptide are administered via oral, rectal, vaginal, topical, nasal, ophthalmic, transdermal, subcutaneous, intramuscular, intraperitoneal or intravenous routes of administration. The route of administration of the pharmaceutical composition will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. Although the bioavailability of peptides administered by other routes can be lower than when administered via parenteral injection, by using appropriate formulations it is envisaged that it will be possible to administer the compositions of the invention via transdermal, oral, rectal, vaginal, topical, nasal, inhalation and ocular modes of treatment. In addition, it may be desirable to introduce the pharmaceutical compositions of the invention by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer.

For topical application, a peptide of the invention, derivative, analog or a fragment thereof can be combined with a pharmaceutically acceptable carrier so that an effective dosage is delivered, based on the desired activity. The carrier can be in the form of, for example, and not by way of limitation, an ointment, cream, gel, paste, foam, aerosol, suppository, pad or gelled stick.

For oral applications, the pharmaceutical composition may be in the form of tablets or capsules, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type, a liquid carrier such as fatty oil. In addition, dosage unit forms can contain various other materials which modify the physical form of the dosage unit, for example, coatings of sugar, shellac, or other enteric agents. The tablets of the invention can further be film coated.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes.

According to some embodiments, the peptide, dimeric peptide, or both, of the invention, can be delivered in a controlled release system. In another embodiment, an infusion pump can be used to administer a peptide such as the one that is used, for example, for delivering insulin or chemotherapy to specific organs or tumors. In another embodiment, the peptide, dimeric peptide, or both, of the invention is administered in combination with a biodegradable, biocompatible polymeric implant, which releases the peptide over a controlled period of time at a selected site. Examples of preferred polymeric materials include, but are not limited to, polyanhydrides, polyorthoesters, polyglycolic acid, polylactic acid, polyethylene vinyl acetate, copolymers and blends thereof (See, Medical applications of controlled release, Langer and Wise (eds.), 1974, CRC Pres., Boca Raton, Fla., the contents of which are hereby incorporated by reference in their entirety). In yet another embodiment, a controlled release system can be placed in proximity to a therapeutic target, thus requiring only a fraction of the systemic dose.

The presently described peptide, may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The compositions also include incorporation of the active material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance.

In one embodiment, it will be appreciated that the peptide, dimeric peptide, or both, of the invention can be provided to the individual with additional active agents to achieve an improved therapeutic effect as compared to treatment with each agent by itself. In another embodiment, measures (e.g., dosing and selection of the complementary agent) are taken to adverse side effects which are associated with combination therapies.

In one embodiment, depending on the severity and responsiveness of the condition to be treated, dosing can be of a single or a plurality of administrations, with course of treatment lasting from several days to several weeks or until cure is affected or diminution of the disease state is achieved.

In some embodiments, the peptide is administered in a therapeutically safe and effective amount. As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the presently described manner. In another embodiment, a therapeutically effective amount of the polypeptide is the amount of the polypeptide necessary for the in vivo measurable expected biological effect. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington: The Science and Practice of Pharmacy, 21st Ed., Lippincott Williams & Wilkins, Philadelphia, Pa., (2005). In some embodiments, preparation of effective amount or dose can be estimated initially from in vitro assays. In one embodiment, a dose can be formulated in animal models and such information can be used to more accurately determine useful doses in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p.1].

Pharmaceutical compositions containing the presently described peptide(s) as the active ingredient can be prepared according to conventional pharmaceutical compounding techniques. See, for example, Remington's Pharmaceutical Sciences, 18th Ed., Mack Publishing Co., Easton, Pa. (1990). See also, Remington: The Science and Practice of Pharmacy, 21st Ed., Lippincott Williams & Wilkins, Philadelphia, Pa. (2005).

In one embodiment, compositions including the preparation of the present invention formulated in a compatible pharmaceutical carrier are prepared, placed in an appropriate container, and labeled for treatment of an indicated condition.

In one embodiment, compositions of the invention are presented in a pack or dispenser device, such as an FDA approved kit, which contains, one or more unit dosages forms containing the active ingredient. In one embodiment, the pack, for example, comprises metal or plastic foil, such as a blister pack. In one embodiment, the pack or dispenser device is accompanied by instructions for administration. In one embodiment, the pack or dispenser is accommodated by a notice associated with the container in a form prescribed by a governmental agency regulating the manufacture, use or sale of pharmaceuticals, which notice is reflective of approval by the agency of the form of the compositions or human or veterinary administration. Such notice, in one embodiment, is labeling approved by the U.S. Food and Drug Administration for prescription drugs or of an approved product insert.

### Methods of Treatment

According to some embodiments, there is provided a method for ameliorating or treating a subject afflicted with cancer or pre-malignancy, the method comprising administering to the subject a therapeutically effective amount of any one of: the peptide of the invention; the dimeric peptide of the invention; and the pharmaceutical composition of the invention, thereby ameliorating or treating the subject afflicted with cancer or pre-malignancy.

According to some embodiments, there is provided a method for reducing deubiquitination activity of a cell, comprising contacting the cell with an effective amount of any one of: the peptide of the invention; the dimeric peptide of the invention; and the pharmaceutical composition of the invention.

According to some embodiments, there is provided a method for inducing or increasing apoptosis rate of a cell, comprising contacting the cell with an effective amount of any one of: the peptide of the invention; the dimeric peptide of the invention; and the pharmaceutical composition of the invention. In some embodiments, the cell is a cancerous cell.

In some embodiments, there present invention is directed to a method for treating, ameliorating, reducing and/or preventing a condition associated with increased deubiquitination activity of a cell in a subject in need thereof, the method comprising the step of: administering to the subject a pharmaceutical composition comprising a therapeutically effective amount of the peptide of the invention, the dimeric peptide of the invention, or both.

In some embodiments, there present invention is directed to a method for treating, ameliorating, reducing and/or preventing a condition associated with increased proliferation activity of a cell in a subject in need thereof, the method comprising the step of: administering to the subject a pharmaceutical composition comprising a therapeutically effective amount of the peptide of the invention; the dimeric peptide of the invention; or both.

In some embodiments, there present invention is directed to a method for treating, ameliorating, reducing and/or preventing a condition associated with increased apoptosis-resistance activity of a cell in a subject in need thereof, the method comprising the step of: administering to the subject a pharmaceutical composition comprising a therapeutically effective amount of the peptide of the invention; the dimeric peptide of the invention, or both.

In some embodiments, the condition associated with: increased deubiquitination activity of a cell, increased proliferation activity of a cell, increased apoptosis-resistance activity of a cell, or any combination thereof, comprises cancer.

As used herein the terms “cancer” or “pre-malignancy” refer to diseases associated with cell proliferation. Non-limiting types of cancer include carcinoma, sarcoma, lymphoma, leukemia, blastoma and germ cells tumors. In one embodiment, carcinoma refers to tumors derived from epithelial cells including but not limited to breast cancer, prostate cancer, lung cancer, pancreas cancer, and colon cancer. In one embodiment, sarcoma refers of tumors derived from mesenchymal cells including but not limited to sarcoma botryoides, chondrosarcoma, Ewing's sarcoma, malignant hemangioendothelioma, malignant schwannoma, osteosarcoma and soft tissue sarcomas. In one embodiment, lymphoma refers to tumors derived from hematopoietic cells that leave the bone marrow and tend to mature in the lymph nodes including but not limited to Hodgkin lymphoma, non-Hodgkin lymphoma, multiple myeloma and immunoproliferative diseases. In one embodiment, leukemia refers to tumors derived from hematopoietic cells that leave the bone marrow and tend to mature in the blood including but not limited to acute lymphoblastic leukemia, chronic lymphocytic leukemia, acute myelogenous leukemia, chronic myelogenous leukemia, hairy cell leukemia, T-cell prolymphocytic leukemia, large granular lymphocytic leukemia and adult T-cell leukemia. In one embodiment, blastoma refers to tumors derived from immature precursor cells or embryonic tissue including but not limited to hepatoblastoma, medulloblastoma, nephroblastoma, neuroblastoma, pancreatoblastoma, pleuropulmonary blastoma, retinoblastoma and glioblastoma-multiforme. In one embodiment, germ cell tumors refer to tumors derived from germ cells including but not limited to germinomatous or seminomatous germ cell tumors (GGCT, SGCT) and nongerminomatous or nonseminomatous germ cell tumors (NGGCT, NSGCT). In one embodiment, germinomatous or seminomatous tumors include but not limited to germinoma, dysgerminoma and seminoma. In one embodiment, non-germinomatous or non-seminomatous tumors refers to pure and mixed germ cells tumors including but not limited to embryonal carcinoma, endodermal sinus tumor, choriocarcinoma, tearoom, polyembryoma, gonadoblastoma and teratocarcinoma.

As used herein, “cancer or pre-malignant cell proliferation” is a molecular process which further to increased cell proliferation rates requires increased deubiquitination activity. In some embodiments, the method of the present invention is directed to reducing deubiquitination activity. In some embodiments, reducing deubiquitination activity results in increased proteasomal activity. In some embodiments, reducing deubiquitination activity results in increased protein degradation. In some embodiments, reducing deubiquitination activity further reduces drug resistance. In some embodiments, a cancerous cell has increased deubiquitination activity compared to a non-cancerous cell or a benign cell. In another embodiment, reducing deubiquitination activity reduces viability of a cancerous cell. In some embodiments, reducing deubiquitination activity increases apoptosis rates in or of a cancerous cell. In some embodiments, increasing cell apoptosis results in reduced cell viability.

In some embodiments, the terms “reduce” or “reducing” used in the abovementioned embodiments (such as for deubiquitination activity of a cell, proteasomal degradation of ubiquitinated proteins, cell viability, or others), are by at least 10%, by at least 20%, by at least 30%, by at least 40%, by at least 50%, by at least 60%, by at least 70%, by at least 80%, by at least 90%, or by at least 100% compared to control, or any value and range therebetween. Each possibility represents a separate embodiment of the invention. In some embodiments, reducing is by 1-5%, 4-10%, 8-20%, 15-30%, 25-40%, 35-55%, 50-70%, 60-80%, 75-90%, 90-99%, or 95-100% compared to control. Each possibility represents a separate embodiment of the invention. In some embodiments, reducing is by at least 2-fold, by at least 3-fold, by at least 5-fold, by at least 10-fold, by at least 15-fold, by at least 20-fold, by at least 40-fold, by at least 75-fold, by at least 100-fold, by at least 150-fold, by at least 200-fold, by at least 500-fold, or by at least 1,000-fold compared to control, or any value and range therebetween. Each possibility represents a separate embodiment of the invention.

The terms “inhibiting”, “reducing” and “decreasing” are interchangeable.

In some embodiments, the term “increase” or “increasing” used in the abovementioned embodiments (such as for pro-apoptotic activity, cell apoptosis rate, or others), is by at least 10%, by at least 20%, by at least 30%, by at least 40%, by at least 50%, by at least 60%, by at least 70%, by at least 80%, by at least 90%, or by at least 100% compared to control, or any value and range therebetween. Each possibility represents a separate embodiment of the invention. In some embodiments, increasing is by 1-5%, 4-10%, 8-20%, 15-30%, 25-40%, 35-55%, 50-70%, 60-80%, 75-90%, 90-99%, or 95-100% compared to control. Each possibility represents a separate embodiment of the invention. In some embodiments, increasing is by at least 2-fold, by at least 3-fold, by at least 5-fold, by at least 10-fold, by at least 15-fold, by at least 20-fold, by at least 40-fold, by at least 75-fold, by at least 100-fold, by at least 150-fold, by at least 200-fold, by at least 500-fold, or by at least 1,000-fold compared to control, or any value and range therebetween. Each possibility represents a separate embodiment of the invention.

In some embodiments, ubiquitination/deubiquitination kinetics or dynamics, are detected by any assay known to in the art, including immune-assays, western-blot, immune-histochemistry, and the like, such as for detecting K48Ub. In some embodiments, protein degradation and proteasomal activity are detected by any acceptable method, including immune-assays, western-blot, immune-histochemistry, pulse-chase assay, and the like, all of which are well known to one of ordinary skill in the art.

The term “subject” as used herein refers to an animal, more particularly to non-human mammals and human organism. Non-human animal subjects may also include prenatal forms of animals, such as, e.g., embryos or fetuses. Non-limiting examples of non-human animals include: horse, cow, camel, goat, sheep, dog, cat, non-human primate, mouse, rat, rabbit, hamster, guinea pig, pig. In one embodiment, the subject is a human. Human subjects may also include fetuses. In one embodiment, a subject in need thereof is a subject afflicted with and/or at risk of being afflicted with a condition associated with increased cell proliferation, deubiquitination activity, or combination thereof.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described peptides prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

As used herein, the term “condition” includes anatomic and physiological deviations from the normal that constitute an impairment of the normal state of the living animal or one of its parts, that interrupts or modifies the performance of the bodily functions.

### Methods of Peptide Identification

According to some embodiments, there is provided a method of identifying a ubiquitin (Ub) high affinity binding peptide, the method comprising the steps: (a) contacting a poly-Ub protein with a peptide suspected of having increased binding affinity to Ub; (b) contacting the poly-Ub protein contacted with the suspected peptide of step (a) with a Ub high affinity cyclic peptide comprising a reporter molecule; and (c) determining a signal generated by the reporter molecule, thereby screening for a Ub high affinity peptide.

As used herein, the term “high binding affinity” is binding with a dissociation constant in the nanomolar scale. In some embodiments, high binding affinity is with a KD ranging from 0.1 nM to 10 nM, 1 nM to 100 nM, 50 nM to 250 nM, 15 nM to 1,500 nM, or 20 nM to 300 nM. Each possibility represents a separate embodiment of the invention.

In some embodiments, a reduced signal compared to a standard signal is indicative of the suspected peptide has increased binding affinity to the poly-Ub protein.

In some embodiments, an increased signal compared to a standard signal is indicative of a suspected peptide has reduced binding affinity to the poly-Ub protein,

In some embodiments, a suspected peptide comprises a plurality of suspected peptides. In some embodiments, a plurality comprises any integer of 2 or greater. In some embodiments, a plurality comprises 2 to 100, 5 to 50, 10 to 1,000, 3 to 75, or 20 to 500. Each possibility represents a separate embodiment of the invention.

In some embodiments, a suspected peptide is a synthetic peptide. In some embodiments, a suspected peptide is extracted or isolated from a cell or a tissue.

In some embodiments, a suspected peptide is linear or cyclic.

In some embodiments, a suspected peptide is unlabeled.

As used herein, the term “unlabeled” refers to the suspected peptide not carrying or conjugated to a moiety capable of generating a detectable signal or being detected.

In some embodiments, the poly-Ub further comprises a molecule having high binding affinity to a capturing protein.

In some embodiments, the molecule is a small molecule. In some embodiments, the molecule is an antigen. In some embodiments, the molecule is a hapten. In some embodiments, the molecule is biotin. In some embodiments, the molecule is specifically bound to the capturing protein.

As used herein, the term “specifically bound” refers to the molecule being inert to any other compound excluding the capturing protein. In some embodiments, the capturing protein binds the molecule with a KD being at least 10-fold lower than the KD representing the binding of the capturing protein with any other compound. In some embodiments, the capturing protein binds primarily, predominantly, or both, to the molecule.

In some embodiments, the capturing protein is streptavidin. In some embodiments, the capturing protein is an antibody.

In some embodiments, the capturing protein is immobilized to a surface. In some embodiments, there is provided a functionalized surface comprising the capturing protein. In some embodiments, immobilized comprises any one of: linked, cross-linked, and chemically bound, e.g., via a covalent bond.

In some embodiments, the method further comprising an additional step comprising removing any one of: (i) a suspected peptide unbound to the poly-Ub, (ii) a Ub high affinity cyclic peptide unbound to the poly-Ub, and/or (iii) a combination of (i) and (ii).

In some embodiments, the additional step is performed between step (b) and step (c) of the method of the invention.

In some embodiments, removing comprises eliminating, washing out, or any equivalent thereof, or any combination thereof, excess of: a suspected peptide, a Ub high affinity cyclic peptide, or both. In some embodiments, “excess” refers to any one of: (i) a suspected peptide unbound to the poly-Ub, (ii) a Ub high affinity cyclic peptide unbound to the poly-Ub, and/or (iii) a combination of (i) and (ii).

In some embodiments, the reporter molecule comprises a fluorescent molecule.

As used herein, the term “fluorescent” encompasses any molecule that emits light after absorbing light or any other electromagnetic radiation. In some embodiments, the reporter molecule is a small molecule. In some embodiments, the reporter molecule is a macromolecule. In some embodiments, the reporter molecule is a peptide or a protein.

In some embodiments, the reporter molecule comprises or consists of fluorescein isothiocyanate (FITC).

As used herein, the term “standard signal” refers to the amount, level, quantity, or any equivalent thereof, of a signal generated by the reporter molecule when applying the herein disclosed method in the absence of a suspected peptide. In some embodiments, the standard signal comprises the signal generated by the reporter molecule when contacting the poly-Ub with the Ub high affinity cyclic peptide in the absence of a suspected peptide.

In some embodiments, the herein disclosed method is directed to the identification of a polypeptide suspected of being capable of binding Ub. In some embodiments, the suspected polypeptide has specific binding affinity to Ub.

As used herein, “Ub high affinity cyclic polypeptide” refers to a cyclic polypeptide comprising 10-30 amino acids and capable of binding to Ub with a binding affinity, e.g., dissociation constant (KD), ranging from 0.1-100 nM. In some embodiments, a Ub high affinity cyclic polypeptide comprises or consists of SEQ ID NO: 13.

Methods for determining binding affinity are common and would be apparent to one of ordinary skill in the art. Non-limiting examples for such methods, include, but are not limited to competitive binding and fluorescent reading, such as exemplified hereinbelow.

Any concentration ranges, percentage range, or ratio range recited herein are to be understood to include concentrations, percentages or ratios of any integer within that range and fractions thereof, such as one tenth and one hundredth of an integer, unless otherwise indicated.

Any number range recited herein relating to any physical feature, such as polymer subunits, size or thickness, are to be understood to include any integer within the recited range, unless otherwise indicated.

As used herein, the terms “subject” or “individual” or “animal” or “patient” or “mammal,” refers to any subject, particularly a mammalian subject, for whom therapy is desired, for example, a human.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include”, and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Generally, the nomenclature used herein, and the laboratory procedures utilized in the present invention include chemical, molecular, biochemical, and cell biology techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); “Cell Biology: A Laboratory Handbook”, Volumes I-III Cellis, J. E., ed. (1994); The Organic Chemistry of Biological Pathways by John McMurry and Tadhg Begley (Roberts and Company, 2005); Organic Chemistry of Enzyme-Catalyzed Reactions by Richard Silverman (Academic Press, 2002); Organic Chemistry (6th Edition) by Leroy “Skip” G Wade; Organic Chemistry by T. W. Graham Solomons and, Craig Fryhle.

### Materials and Methods

### General Methods

Solid Phase Peptide Synthesis (SPPS) was carried out manually in syringes, equipped with teflon filters, purchased from Torviq or by using an automated peptide synthesizer (CS336X, CSBIO). Unless mentioned, all the chemicals are analytical grade. N, N-dimethylformamide (DMF), N, N-Diisopropylethylamine (DIEA), trifluoroacetic acid (TFA) and dichloromethane (DCM) were purchased from Biolab. Triisopropylsilane (TIPS), ammonium carbonate (NH₄)₂CO₃ and dithiothreitol (DTT) were purchased from Alfa Aesar. Dimethyl sulfoxide(DMSO), Palladium (II) chloride (PdCl₂), HEPES (4-(2-Hydroxyethyl)piperazine-1-ethanesulfonic acid, Iodoacetamide, Benzyl bromide (Reagent grade), 2-(Bromomethyl)naphthalene, 2-bromoethan-1-aminehydro bromide, 4-grade) bromomethyl-7-methoxycoumarin, Iodomethane (Reagent and Hexafluorobenzene (Reagent grade) were purchased from Sigma-Aldrich. Fluorescein-5-Maleimide was purchased from Thermo Fisher Scientific. 2-Chloroacetic acid was purchased from Acros Organics. Resins were purchased from CreoSalus, 9-fluorenylmethoxycarbonyl (Fmoc) and tert-Butyloxycarbonyl (Boc) protected amino acids were purchased from GL Biochem. Activating reagents 1-[Bis(dimethylamino)methylene]-1H-1,2,3-triazolo[4,5-b]pyridinium 3-oxid hexafluorophosphate (HATU)], [(2-(1H-benzotriazol-1-yl)-1,1,3,3-tetramethyluronium hexafluorophosphate (HBTU), [(6-chlorobenzotriazol-1-yl)oxy-(dimethylamino)methylidene]-dimethylazanium hexafluorophosphate (HCTU) and hydroxybenzotriazole (HOBt) were purchased from Luxembourg Bio Technologies and GL Biochem. Analytical high-performance liquid chromatography (HPLC) was performed on a Thermo instrument (Dionex Ultimate 3000) using analytical XSelect (waters, CSH C18, 3.5 μm, 4.6×150 mm) and Xbridge (waters, BEH300 C4, 3.5 μm, 4.6×150 mm) columns at flow rate of 1.2 ml/min. Semi preparative HPLC was performed on a Thermo Scientific instrument (Dionex Ultimate 3000) using Jupiter C18 (Phenomenex, 10 μm, 300 Å, 250×10 mm) column and Jupiter C4 (Phenomenex, 10 μm, 300 Å, 250×10 mm) column, at flow rate of 4 mL/min. Preparative HPLC was performed on a Thermo Scientific instrument (Dionex Ultimate 3000) using XSelect (waters, CSH C18, 10 μm, 19×250 mm), Jupiter C18 (Phenomenex, 5 μm, 300 Å, 250×21.2 mm) and Jupiter C4 (Phenomenex, 10 μm, 300 Å, 250×22.4 mm) column, at flow rate of 15 mL/min. Following HPLC step, the chemically synthesized biotin-tetraUbK48 was further purified using the AKTA fast protein liquid chromatography (FPLC) system (GE). All synthetic products were purified by HPLC and characterized by mass spectrometry. Mass spectrometry: Electrospray ionization mass spectrometry (ESI-MS) was performed on a LCQ Fleet mass spectrometer (Thermo Scientific) with an ESI source. All calculated masses have been reported as an average isotope composition. Fluorescent measurements were performed on an Infinite M200 fluorescence plate reader (TECAN). HPLC mobile phases, Buffer A: 0.1% TFA in water; buffer B: 0.1% TFA in acetonitrile. HEPES Buffer (50 mM HEPES, NaCl (150 mM), 0.1% Tween, pH=7.3).

### Cells

HeLa cells (CCL-2™) were purchased from ATCC®. Dulbecco's modified Eagle's medium (DMEM), Eagle's minimum essential medium (EMEM), Fetal bovine serum (FBS), L-Glutamine, antibiotics (penicillin/streptomycin), trypsin/EDTA and phosphate-buffered saline (PBS) were purchased from biological industries. Hoechst 33342 solution (20 mM), SYTOX blue (5 mM) and Lysotracker probes were purchased from Thermo-fisher. μ-Slide 8 well ibiTreat for live-cell confocal microscopy was purchased from ibidi and polylysine hydrobromide was purchased from Sigma. Distribution of fluorescent peptides in live cells was analyzed using a confocal laser scanning microscope (Confocal Zeiss LSM 710) equipped with 40×NA 1.2 water immersion objective. During CLSM analysis the samples were kept at 37° C. in a humidified chamber.

### Fluorescence-Based Competitive Assay for Peptide Screening

Streptavidin-coated microplate (96 well plate) was washed (3×100 μl) with HEPES buffer. Then, 1 μg of biotin-tetra-UbK48 in 100 μl of HEPES buffer was incubated in each well for 30 minutes at room temperature (RT). Additional wells were kept free of biotin-tetra-UbK48 for subsequent blank subtraction. Following a washing step, unlabeled candidate peptides, including peptide-standard (Ub4_ix), were incubated in an excess amount (5 molar equiv. relative to biotin-tetra-UbK48) for 30 minutes at RT to saturate binding to the target. Next, following a washing step FITC-labeled peptide-standard (Ub4_ix-FITC) was incubated for 30 minutes at RT in 1 molar equivalent relative to biotin-tetra-UbK48 to compete with the unlabeled peptides. Finally, following washing, each well was treated with 100 μl of 6 M Gn·HCl at RT for 30 minutes to release the bound peptides. Then, the solution with the released peptides was transferred to Nunc black 96-well plate and fluorescence was measured (λ excitation=480 nm and λ emission=525 nm). The obtained values were then normalized, and compared with peptide standard, while the fluorescence signal was inversely proportional to the binding affinity of the peptide. The calculations of relative change in binding were performed according to the formula: Y=−[1−(a/b)]×100, where Y is the change in the signal relative to the standard Ub4_ix (in percentage); a is signal measured for each peptide variant (in relative fluorescence units); b is signal measured for Ub4_ix.

To determine the dissociation constant (Kd) of labeled peptides, following immobilization step of biotin-tetra-UbK48, different concentrations of the labeled peptide were incubated. Then, the bound cyclic peptides were released with 6 M Gn·HCl, followed by fluorescence measurement (λex=480 nm and λem=525 nm). After normalization, binding curves were obtained and used for kinetic determination of Kd.

### Synthesis of Cyclic Peptides

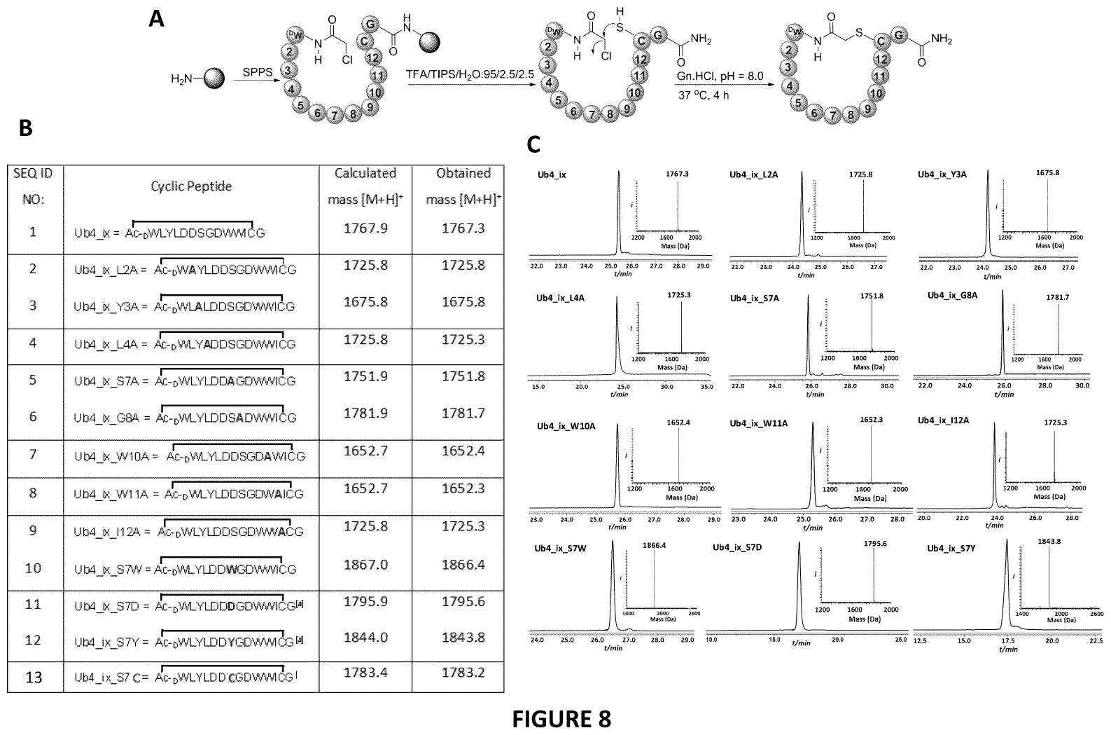

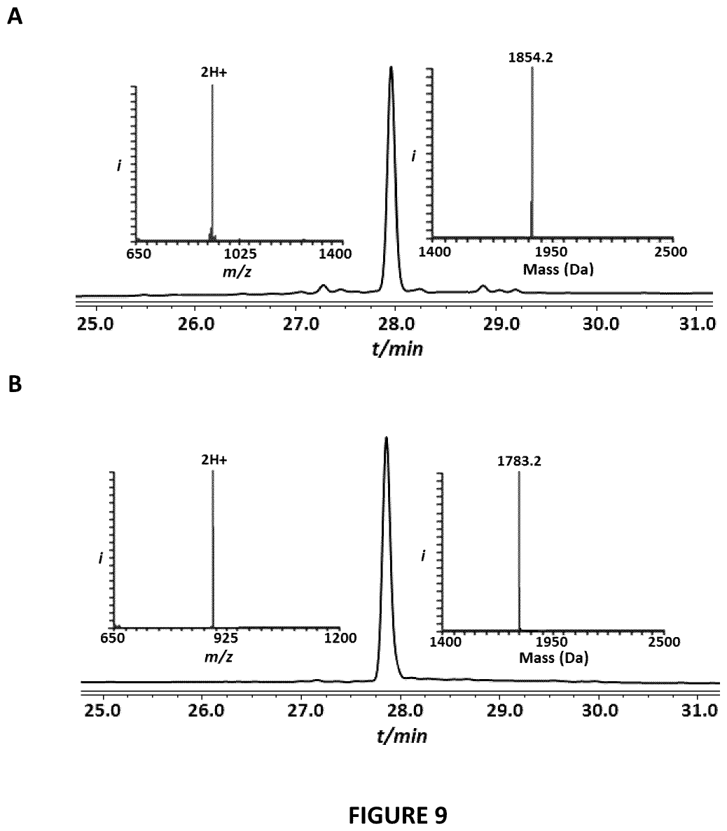

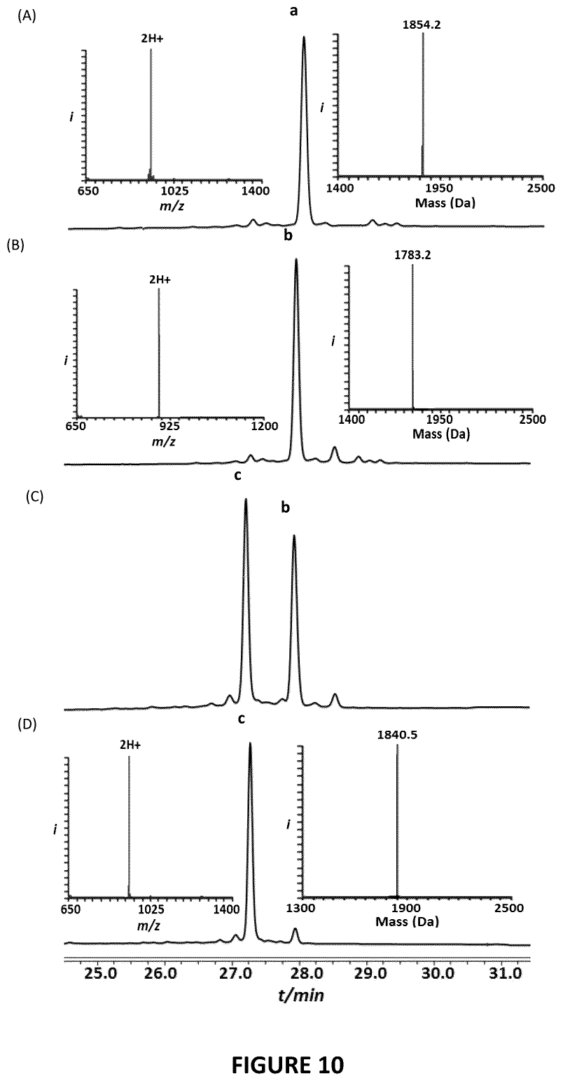

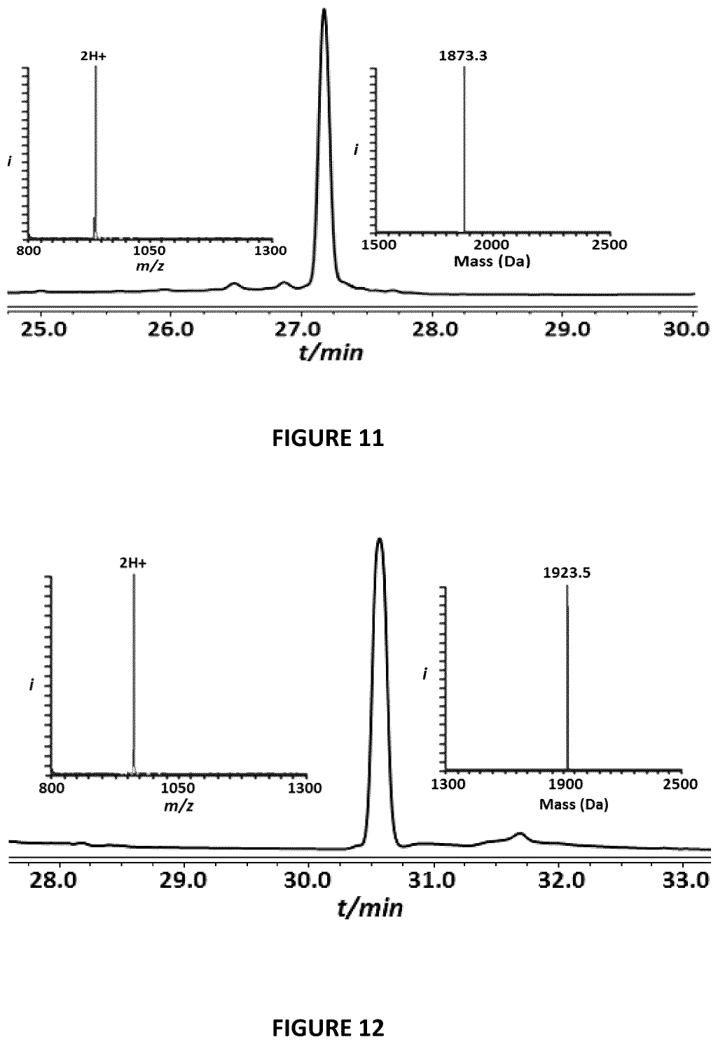

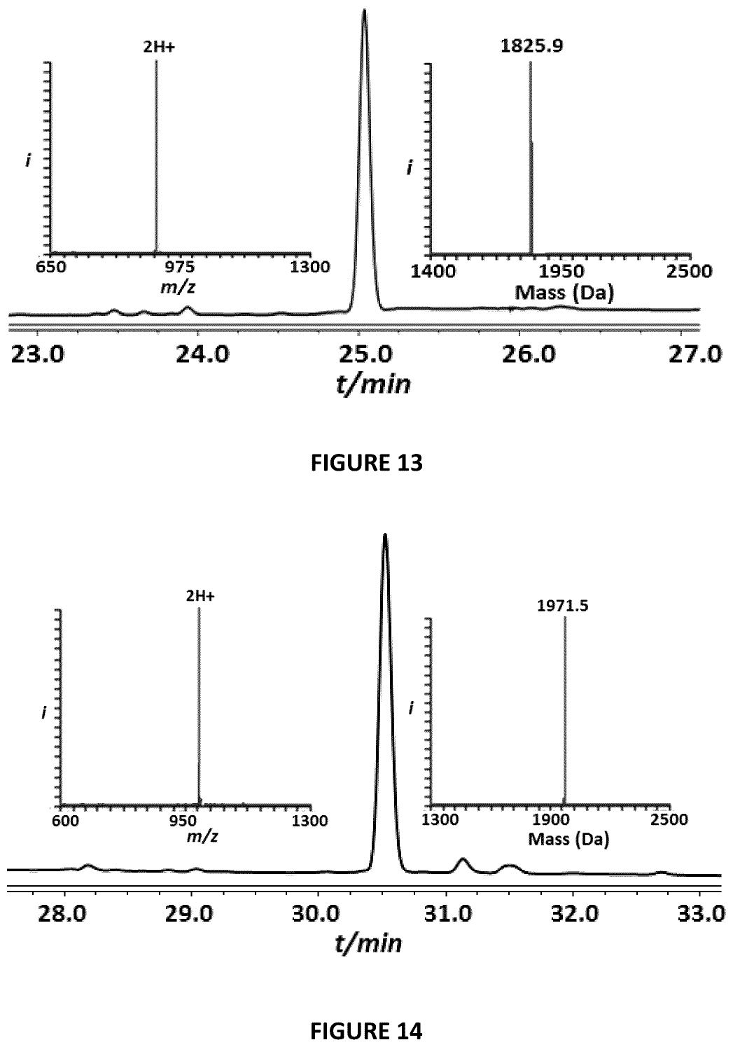

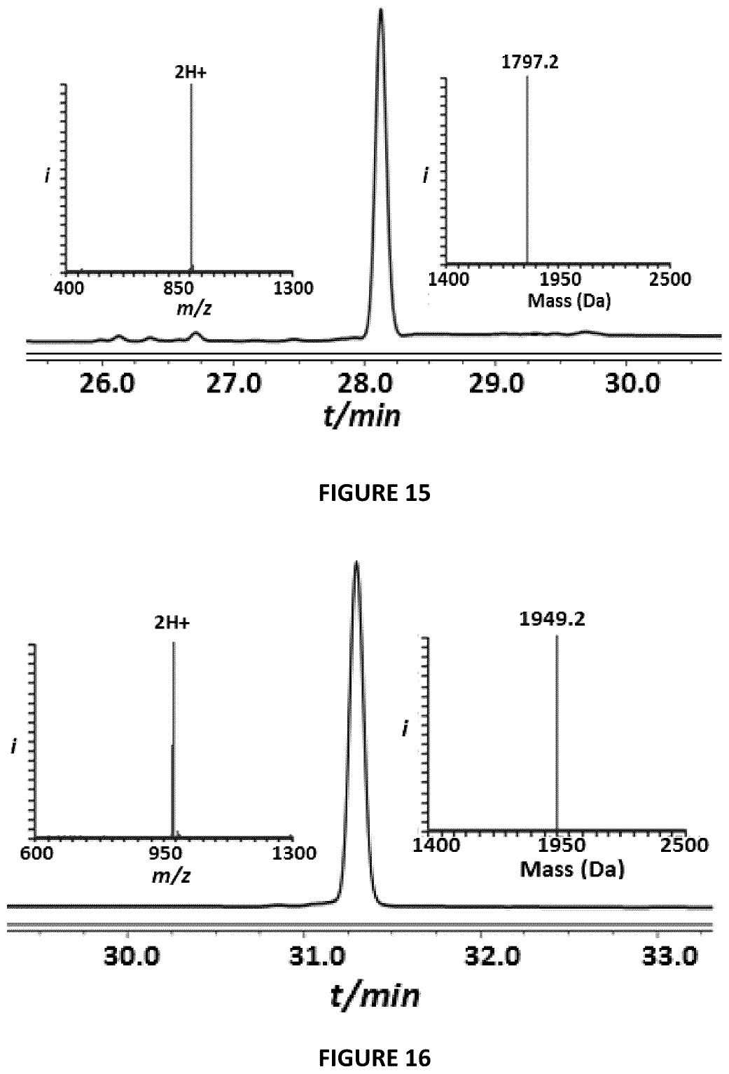

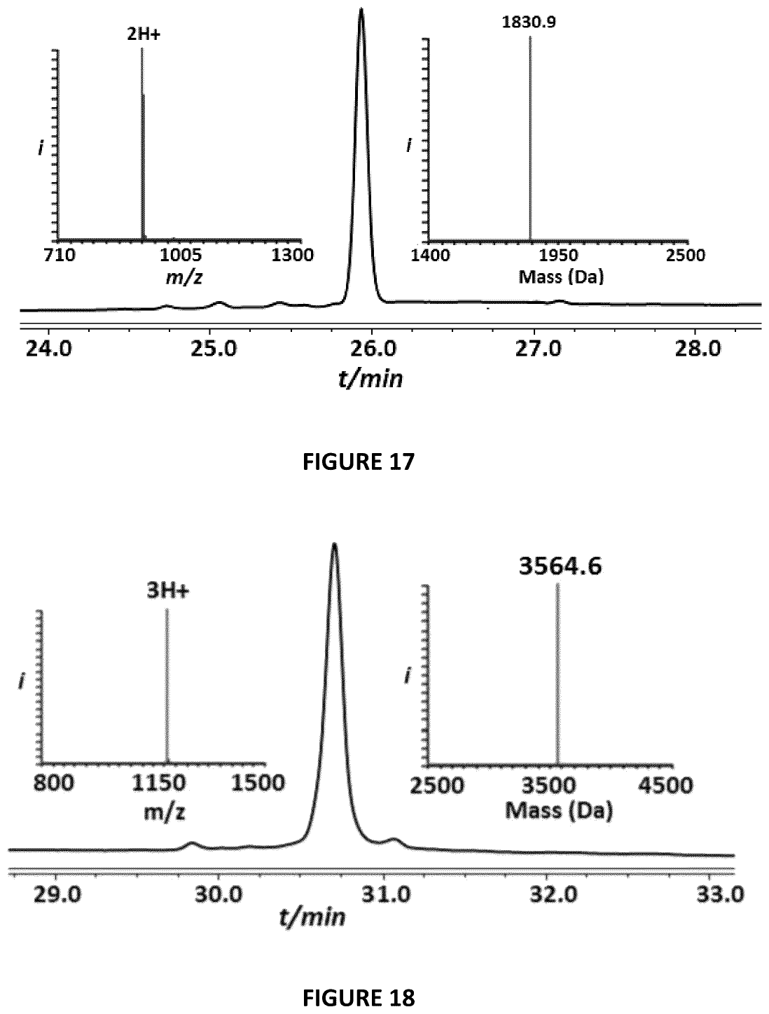

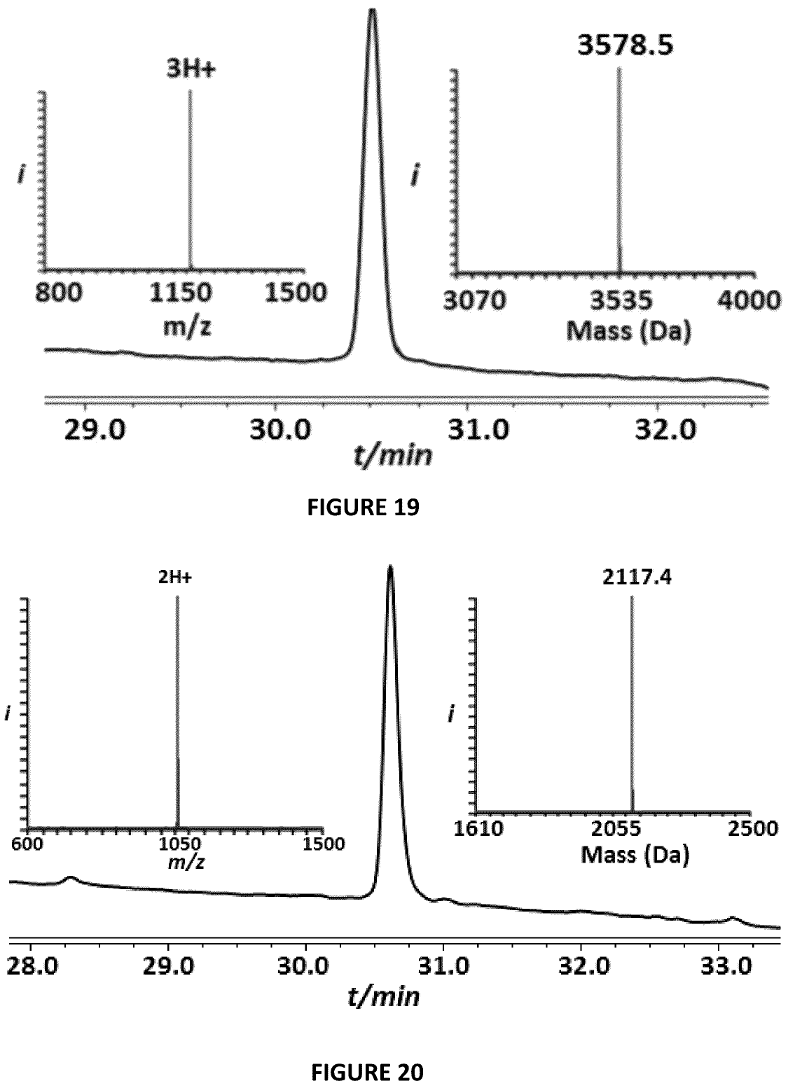

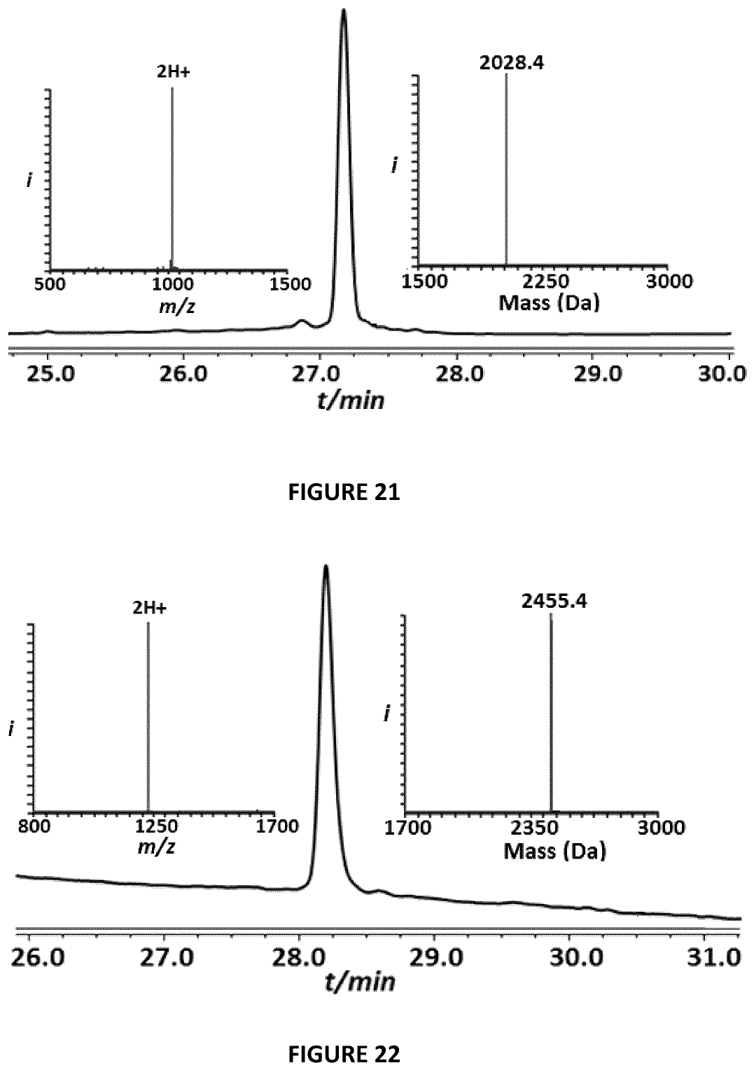

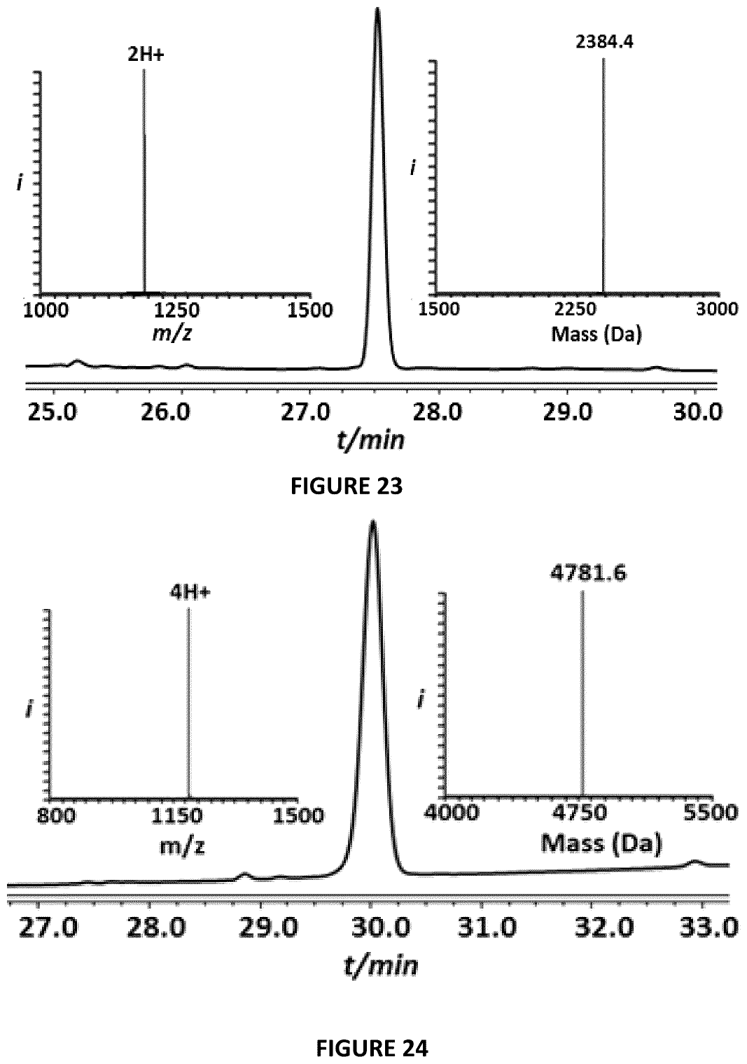

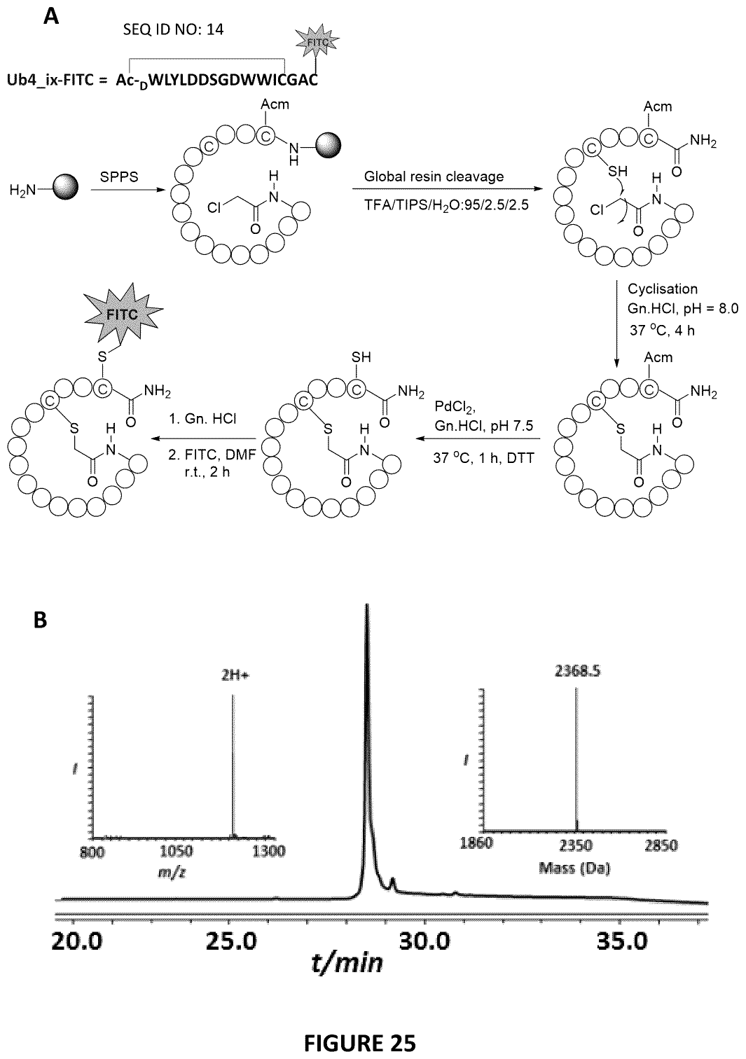

Cyclic peptides were synthesized using Fmoc-SPPS as summarized in FIG. 8A. Chloroacetic acid was coupled at N-terminal position. Then the peptide was cleaved from the resin using the mixture of TFA/H₂O/TIS (95:2.5:2.5) followed by precipitation in cold diethyl ether and lyophilization. The crude peptides were dissolved in 6M Gn·HCl followed by incubation at 37° C. pH 8 for 4h for cyclization. The crude peptide was purified by HPLC employing a C18 column with a gradient flow of 0-60% B in 60 min. All analogues of cyclic peptides were synthesized in a similar manner.

### Synthesis of Cyclic Peptide 14, Ub4_ix_S7C

To synthesize cyclic peptide Ub4_ix_S7C an orthogonally Acetamido methyl (Acm) protected Cys7 was incorporated to the Ub4_ix scaffold, following by cleavage from the resin and subsequent cyclization to give cyclic peptide 13. Next, for the Acm removal, the peptide (10.0 mg, 5.39×10⁻³ mmol, 1.0 equiv) was dissolved in argon purged 6 M Gn·HCl/200 mM phosphate buffer (pH 7.5, 2,695 μl, 2 mM). Then, PdCl₂ (9.54 mg, 10.0 equiv) dissolved in 100 μl 6 M Gn·HCl/200 mM phosphate buffer (pH 7.5) was added to the peptide solution and the reaction mixture was incubated at 37° C. for 1 h. Finally, the reaction mixture was quenched with the dithiothreitol (DTT), (40.0 equiv, 2.15×10⁻¹ mmol), followed by centrifugation and injection of the supernatant into HPLC using a semi-preparative C18 column with a gradient flow of 0-60% B in 60 min to give the purified Ub4_ix_S7C (4.04 mg, 42% yield) with the free thiol.

### Synthesis of Cys-Modified Cyclic Peptides

Ub4_ix_S7C was further subjected to alkylation, arylation and oxidation for making various Cys-modified cyclic peptides, which are described as follows:

(a) One-Pot Synthesis for Cysteine-Alkylation, a(i)

The inventors developed a one-pot strategy for synthesizing the various cysteine-alkylated peptide derivatives from the Cys(Acm) protected cyclic peptide (Ub4_ix_S7C(Acm)). The synthesis of each derivative was described as follows.

(i) Synthesis of Cyclic Peptide 16, Ub4_ix_S7C-CH₂CONH₂

Various Cys-alkylated derivatives were synthesized from Ub4_ix_S7C(Acm) cyclic peptide in one pot manner. Acm removal was performed as previously described. After quenching the reaction mixture followed by centrifugation, the supernatant was separated, and the pH adjusted to 8.0. Then 500 equiv of 2-iodoacetamide dissolved in 500 μl of DMF was added to the reaction mixture at room temperature. The progress of the reaction was monitored by HPLC using a C18 analytical column with a gradient of 0-60% B in 30 min. After completion of reaction (4.5 h), the mixture was purified using a preparative C18 column with a gradient flow of 0-60% B in 60 min give Ub4_ix_S7C-CH₂CONH₂ (5.86 mg, 59% yield).

(ii) Synthesis of Cyclic Peptide 15, Ub4_ix_S7C-CH₂C₆H₅

The peptide Ub4_ix_S7C(Acm) (10.0 mg, 5.39×10⁻³ mmol, 1.0 equiv) was subjected to Acm removal as previously described. After quenching the reaction mixture followed by centrifugation, the supernatant was separated and adjusted pH to 8.0 with NaOH. Subsequently, 500 equiv of benzyl bromide dissolved in 500 μl of DMF was added to the reaction mixture at room temperature. The progress of the reaction was monitored by HPLC using a C4 analytical column with a gradient of 0-60% B in 30 min. After completion of reaction, (7 h), the mixture was purified using a preparative C4 column with a gradient flow of 0-60% B in 60 min give Ub4_ix_S7C-CH₂C₆H₅ (5.05 mg, 50% yield).

(iii) Synthesis of Cyclic Peptide 17, Ub4_ix_S7C-CH₂C₁₀H₇

The peptide Ub4_ix_S7C(Acm) (10.0 mg, 5.39×10⁻³ mmol, 1.0 equiv) was subjected to Acm removal as previously described. After quenching the reaction mixture followed by centrifugation, the supernatant was separated and adjusted pH to 8.0 with NaOH. Then 500 equiv of 2-(bromomethyl)naphthalene dissolved in 500 μl of DMF was added to the reaction mixture at room temperature. The progress of the reaction was monitored by HPLC using a C18 analytical column with a gradient of 0-60% B in 30 min. After completion of reaction (7 h), the reaction mixture was purified using a preparative C18 column with a gradient flow of 0-60% B in 60 min give Ub4_ix_S7C-CH₂C₁₀H₇ (4.88 mg, 47% yield).

(iv) Synthesis of Cyclic Peptide 18, Ub4_ix_S7C-CH₂CH₂NH₂

The peptide Ub4_ix_S7C(Acm) (10.0 mg, 5.39×10⁻³ mmol, 1.0 equiv) was subjected to Acm removal as previously described. After quenching the reaction mixture followed by centrifugation, the supernatant was separated and adjusted pH to 8.0 with NaOH. Then 500 equiv of 2-bromoethan-1-amine·HBr dissolved in 500 μl of DMF was added to the reaction mixture at room temperature. The progress of the reaction was monitored by HPLC using a C4 analytical column with a gradient of 0-60% B in 30 min. After completion of reaction, 7 h, the reaction mixture was purified using a preparative C4 column with a gradient flow of 0-60% B in 60 min to giveUb4_ix_S7C-CH₂CH₂NH₂(4.53 mg, 46% yield).

### (v) Synthesis of Cyclic Peptide 19, Ub4_ix_S7C-CH₂C1011703

The peptide Ub4_ix_S7C(Acm) (10.0 mg, 5.39×10⁻³ mmol, 1.0 equiv) was subjected to Acm removal as previously described. After quenching the reaction mixture followed by centrifugation, the supernatant was separated and adjusted pH to 8.0 with NaOH. Then 500 equiv of 4-bromomethyl-7-methoxycoumarin dissolved in 500 μl of DMF was added to the reaction mixture at room temperature. The progress of the reaction was monitored by HPLC using a C18 analytical column with a gradient of 0-60% B in 30 min. After completion of reaction, 7 h, the reaction mixture was purified using a preparative C18 column with a gradient flow of 0-60% B in 60 min give Ub4_ix_S7C-CH₂C₁₀H₇O₃(4.89 mg, 46% yield).

### (vi) Synthesis of Cyclic Peptide 20, Ub4_ix_S7C-CH₃

The peptide Ub4_ix_S7C(Acm) (10.0 mg, 5.39×10⁻³ mmol, 1.0 equiv) was subjected to Acm removal according to the procedure described in section 4. After quenching the reaction mixture followed by centrifugation, the supernatant was separated and adjusted pH to 8.0 with NaOH. Then 500 equiv of iodomethane dissolved in 500 μl of DMF was added to the reaction mixture at room temperature. The progress of the reaction was monitored by HPLC using a C18 analytical column with a gradient of 0-60% B in 30 min. After completion of reaction, 4 h, the reaction mixture was purified using a preparative C18 column with a gradient flow of 0-60% B in 60 min give Ub4_ix_S7C-CH₃(4.85 mg, 50% yield).

Cysteine-Acylation, a(ii)
(vii) Synthesis of Cyclic Peptide 21, Ub4_ix_S7C-C₆F₅

The peptide Ub4_ix_S7C (10.0 mg, 5.61×10⁻³ mmol, 1.0 equiv) was dissolved in argon purged 2.8 mL of 50 mM solution of TRIS base in DMF. Then hexafluoro benzene (6.5 μl, 10.0 equiv) dissolved in 100 μl of DMF was added to the peptide solution. The reaction mixture was vigorously mixed for 25 seconds and left the reaction at room temperature. The progress of the reaction was monitored by HPLC using a C18 analytical column with a gradient of 0-60% B in 30 min. After completion of reaction, 5 h, the reaction mixture was purified using a preparative C18 column with a gradient flow of 0-60% B in 60 min give Ub4_ix_S7C-C₆F₅ (4.70 mg, 43% yield).

### (b) Synthesis of Cyclic Peptide 22, Ub4_ix_S7C(SO₃H)

Ub4_ix_S7C (10.0 mg, 5.6×10⁻³ mmol) was dissolved in 5% acetic acid in water (3,737 μl, 1.5 mM). The solution was adjusted to pH 6 with (NH₄)₂CO₃, following addition of DMSO (374 μl, 5% by volume). During the reaction oxygen (O₂) gas was purged continuously into the solution at room temperature. The progress of the reaction was monitored by HPLC using a C18 analytical column with a gradient of 0-60% B for 30 min and found that the reaction was completed in 6 h. The reaction mixture was purified using a semi-preparative C18 column with a gradient flow of 0-60% B in 60 min give Ub4_ix_S7C(SO₃H) (3.90 mg, 38% yield).

Synthesis of Dimer with Disulfide Bond 23, (Ub4_ix_S7C)₂

Peptide Ub4_ix_S7C (10.0 mg, 5.61×10⁻³ mmol, 1.0 equiv) was dissolved in CH₃COOH:H₂O (4:1 v/v/2242:561 μl), to a final concentration of 2 mM. Then, iodine (˜1.83 mg, 2.0 equiv) was added to the peptide solution in one portion. The reaction mixture was incubated at 37° C. The progress of the reaction was monitored by HPLC using a C18 analytical column with a gradient of 0-60% B in 30 min. After completion of reaction (overnight) the mixture was quenched with twice the volume of water and extracted iodine with CH₂Cl₂ (4-5 times). The aqueous portion was lyophilized, and the product was purified using a semi-preparative C18 column with a gradient flow of 0-60% B in 60 min give dimer with disulfide bond, (Ub4_ix_S7C)₂, (1.15 mg, 23% yield).

Synthesis of Staple Dimer 24, (Ub4_ix_S7C)₂CH₂

The peptide Ub4_ix_S7C (10.0 mg, 5.61×10⁻³ mmol, 1.0 equiv) was dissolved in H₂O (4.0 mM). A solution of TCEP (0.5 equiv) dissolved in 62 μl of H₂O was added to the peptide solution. Then, triethylamine (15.0 equiv) in 85 μl of THF and diiodomethane (12.0 equiv) in 55 μl of THF were sequentially added to the peptide solution at room temperature. The progress of the reaction was monitored by HPLC using a C18 analytical column with a gradient of 0-60% B in 30 min. After completion of reaction (48 h), the mixture was diluted with H₂O (8.0 mL) and lyophilized. Then, the crude peptide was purified using a semi-preparative C18 column with a gradient flow of 0-60% B in 60 min to give stapled dimer, (Ub4_ix_S7C)₂CH₂, (0.90 mg, 18% yield).

### Synthesis of Cyclic Peptide 26

The Synthesis of peptide 25 was carried out using Fmoc-SPPS on a Rink amide resin (0.26 mmol/g, 0.1 mmol scale). Peptide synthesis was carried out in the presence of amino acid (4.0 equiv), HCTU (4.0 equiv) and DIEA (8.0 equiv) at room temperature. The pre-swollen resin was treated with 20% piperidine in DMF containing 0.1 mmol HOBt (3:5:3 min) to remove the Fmoc protecting group. The amino acids were coupled on an automated peptide synthesizer. In order to synthesize FITC-labeled stapled dimer, cysteine 7 and 16 were orthogonally protected with Acm and —S^{t}Bu, respectively, for later stage modifications. To the N-terminus, 2-chloroacetic acid was coupled. After completion of synthesis the resin was washed with DMF, MeOH, DCM and dried under vacuum. After, global deprotection was performed using TFA:TIS:H₂O (95:2.5:2.5) cocktail for 2 h. The cleavage mixture was filtered, and the combined filtrate was added drop wise to a 5-fold volume of cold ether and centrifuged. The precipitated crude peptide 25 was dissolved in ACN:H₂O (1:1) and was further diluted to ˜30% with water and lyophilized. The cyclization reaction was performed by dissolving crude peptide 25 (4.0 mM) in 6 M Gn·HCl/200 mM phosphate buffer (pH 7.5), adjusted to pH 8.0 with NaOH and incubated at 37° C. The progress of the reaction was monitored by HPLC using a C18 analytical column with a gradient of 0-60% B in 30 min. After completion of reaction, 4 h, the crude peptide was purified using a preparative C18 column with a gradient flow of 0-60% B in 60 min to give peptide 26 in 47% yield.

### Synthesis of Cyclic Peptide 27

Peptide 26 (10.0 mg, 4.72×10⁻³ mmol, 1.0 equiv) was dissolved in argon purged 6 M Gn·HCl/200 mM phosphate buffer (pH 7.5, 2360 p1, 2 mM). A solution of TCEP (50.0 equiv) in H₂O was added to the peptide solution following adjusted to pH 2.5 and incubated at 37° C. The progress of the reaction was monitored by HPLC using a C18 analytical column with a gradient of 0-60% B in 30 min. After completion of reaction, 4 h, the crude peptide was filtered and purified using a semi-preparative C18 column with a gradient flow of 0-60% B in 60 min give peptide 27 (4.02 mg, 42% yield).

### Synthesis of Cyclic Peptide 28

Peptide 27 (10.0 mg, 4.93×10⁻³ mmol, 1.0 equiv) was dissolved in argon purged 6 M Gn·HCl/200 mM phosphate buffer (pH 7.5, 2,695 p1, 2 mM). A solution of fluorescein-5-maleimide (6.31 mg, 3.0 equiv) in 50 p.1 of DMF was added to the peptide at room temperature under dark conditions. The progress of the reaction was monitored by HPLC using a C18 analytical column with a gradient of 0-60% B in 30 min. After completion of reaction, 2 h, the crude peptide was filtered and purified using a semi-preparative C18 column with a gradient flow of 0-60% B in 60 min give peptide 28 (4.84 mg, 40% yield).

### Synthesis of Cyclic Peptide 29

Peptide 28 (10.0 mg, 4.07×10⁻³ mmol, 1.0 equiv) was dissolved in argon purged 6 M Gn·HCl/200 mM phosphate buffer (pH 7.5, ˜2036 μl, 2 mM). Then PdCl₂ (˜1.44 mg, 2.0 equiv) dissolved in 50 μl of argon purged 6 M Gn·HCl/200 mM phosphate buffer (pH 7.5) at 37° C. for 10 min was added to the peptide solution and adjusted to pH 1.5, and kept the reaction at room temperature. The progress of the reaction was monitored by HPLC using a C18 analytical column with a gradient of 0-60% B in 30 min. After completion of reaction, 30 min, the reaction mixture was quenched with the dithiothreitol, DTT, (8.0 equiv, 3.26×10⁻² mmol), followed by centrifugation and injection of the supernatant into HPLC using a semi-preparative C18 column with a gradient flow of 0-60% B in 60 min give peptide 29 (1.94 mg, 20% yield) with free thiol.

Synthesis of FITC Labeled Stapled Dimer 30, (Ub4_ix_S7C)₂CH₂-FITC

Peptide 29 (10.0 mg, 4.19×10⁻³ mmol, 1.0 equiv) was dissolved in H₂O (4.0 mM). TCEP (0.5 equiv) dissolved in 55 μl of H₂O was added to the peptide solution. Then, triethylamine (15.0 equiv) in 75 μl of THF and diiodomethane (12.0 equiv) in 50 μl of THF were sequentially added at room temperature. The progress of the reaction was monitored by HPLC using a C4 analytical column with a gradient of 0-60% B in 30 min. After completion of reaction, 48 h, the reaction mixture was diluted with H₂O (8.0 mL) and lyophilized. Then, the crude peptide was purified using a semi-preparative C4 column with a gradient flow of 0-60% B in 60 min give stapled dimer 30, (Ub4_ix_S7C)₂CH₂-FITC (0.60 mg, 12% yield).

### Synthesis of Ub4_ix-FITC

For synthesizing FITC labeled cyclic peptide, a similar strategy as described in FIG. 8A was employed. However, Fmoc-Cys(Acm) was coupled as the first amino acid for a late stage modification. After cyclization step, Cys(Acm) was unmasked by adding 10 equiv of PdCl₂ in 6 M Gn·HCl/200 mM phosphate buffer (pH 7.5) to the peptide (2 mM) in the same buffer and incubated at 37° C. for 30 min. In the following step, the peptide with the free thiol was dissolved in argon purged 6 M Gn·HCl/200 mM phosphate buffer (pH 7.5, 2 mM). A solution of fluorescein-5-maleimide (3.0 equiv) in DMF was added to the peptide at room temperature under dark conditions. The progress of the reaction was monitored by HPLC using a C4 analytical column with a gradient of 0-60% B in 30 min. After completion of reaction, 2 h, the crude peptide was filtered and purified by HPLC employing C4 column and gradient 0-60% B in 60 minutes.

### K_{d} Determination

### (i) K_{d} Determination of Ub4_ix-FITC

The K_{d} value for the FITC labeled Ub4_ix was calculated as previously described. After normalization, the curve of fluorescence versus concentration was generated using Sigma Plot. The dissociation constant was determined to be 35.09±2.66 nM.

K_{d} Determination for 30, (Ub4_ix_S7C)₂CH₂-FITC

The K_{d} value for the FITC labeled dimeric peptide 30 was calculated as previously described and found to be 12.32±1.26 nM.

### Flow Cytometry

Induction of apoptosis in Hela cell lines by treatment with cyclic peptides was determined using annexin V-FITC/propidium iodide(PI) apoptosis detection kit (BD Biosciences) according to the manufacturer's protocol. 2×10⁵ cells/well were treated with inhibitors for 24- and 48-hours in a dose dependent manner. Apoptotic cells were determined as annexin V-FITC positive and PI negative. DMSO and MG132 were used as negative and positive controls respectively. Samples were acquired on a Becton Dickinson LSR II flow cytometer (BD Biosciences) and analyzed using Flowjo software.

### Peptide Cell Uptake

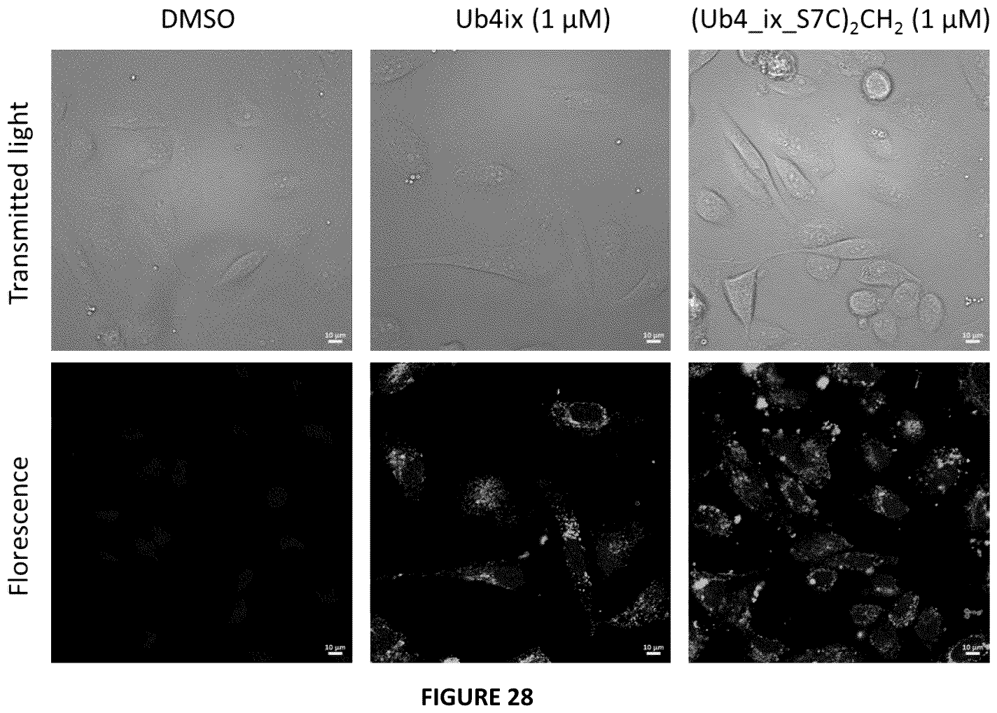

HeLa cells were seeded to 80% confluency on poly-L-Lysine treated slides and incubated in the presence of either DMSO, Ub4_ix-FITC or (Ub4_ix_S7C)₂CH₂-FITC for the indicated times. The cells were washed with PBS buffer followed by addition of fresh phenol-free optical medium and live-cell imaging was carried out using a LSM 710 laser scanning confocal microscope equipped with an environmental control module. (Ub4_ix_S7C)₂CH₂-FITC was quickly and efficiently distributed within cells (FIG. 28), the labeled peptide has successfully entered the cytoplasm and the nucleus.

### Example 1

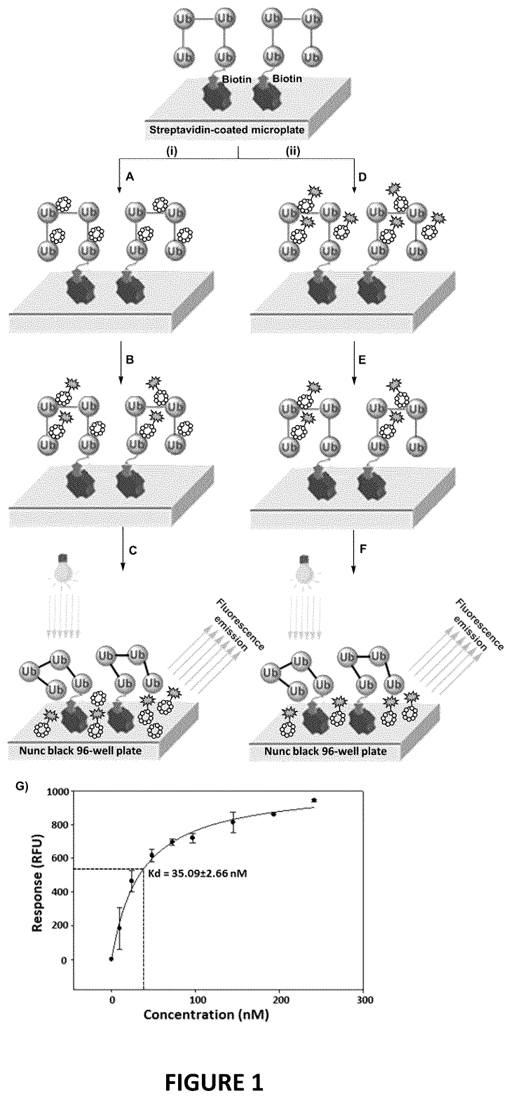

The inventors envisioned a screening strategy to proceed through four main steps, each terminated by washings with binding buffer. First, biotin tagged tetra-UbK48 was immobilized in a constant amount (1 μg per well) in streptavidin covered 96 well plate (FIG. 1(i and ii)). Then, unlabeled candidate peptides were incubated in excess in each well to saturate binding to the target. Notably, unlabeled peptide-standard (Ub4_ix) with known binding parameters was also introduced in one of the wells for the subsequent calculation of relative affinity. Next, fluorescein-5-maleimide (FITC) labeled peptide-standard (Ub4_ix-FITC) was introduced in equal molar ratio to the target (tetra-Ub) in order to compete with the unlabeled candidate peptides. Finally, the bound peptides were released from wells by treatment with 6 M guanidine hydrochloride (Gn·HCl) solution followed by measuring the gained fluorescence (FIG. 1(i)). The obtained values were then compared to the fluorescence of the control well containing the unlabeled peptide to calculate relative binding. The gained fluorescence was inversely proportional to the binding affinity of the peptide candidate. In addition to measuring the relative binding, the herein disclosed protocol can be also slightly modified to determine a dissociation constant (K_{d}) of labeled peptides. For this purpose, after the immobilization step of tetra-Ub, different concentrations of the labeled peptides were incubated, followed by release with 6 M Gn·HCl and fluorescence measurement (FIG. 1(ii)) to plot a binding curve and calculate the dissociation constant (FIG. 1G).

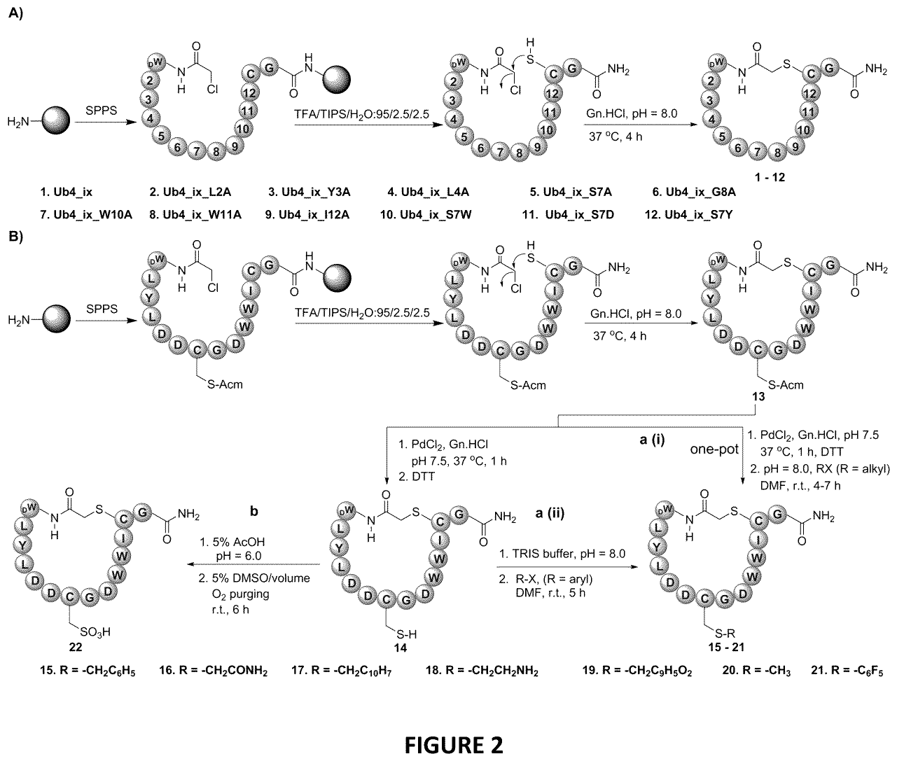

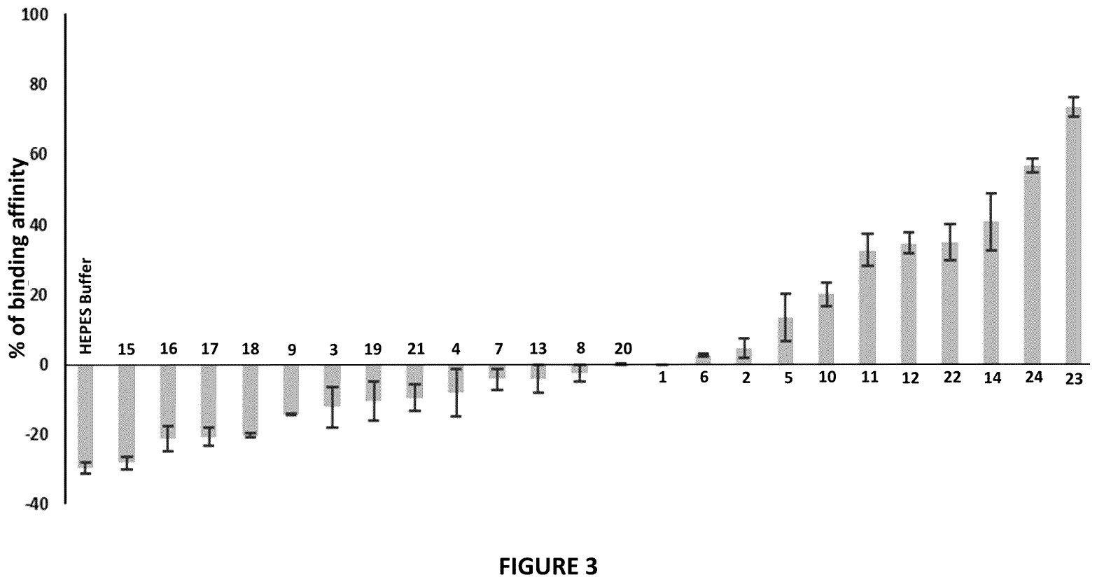

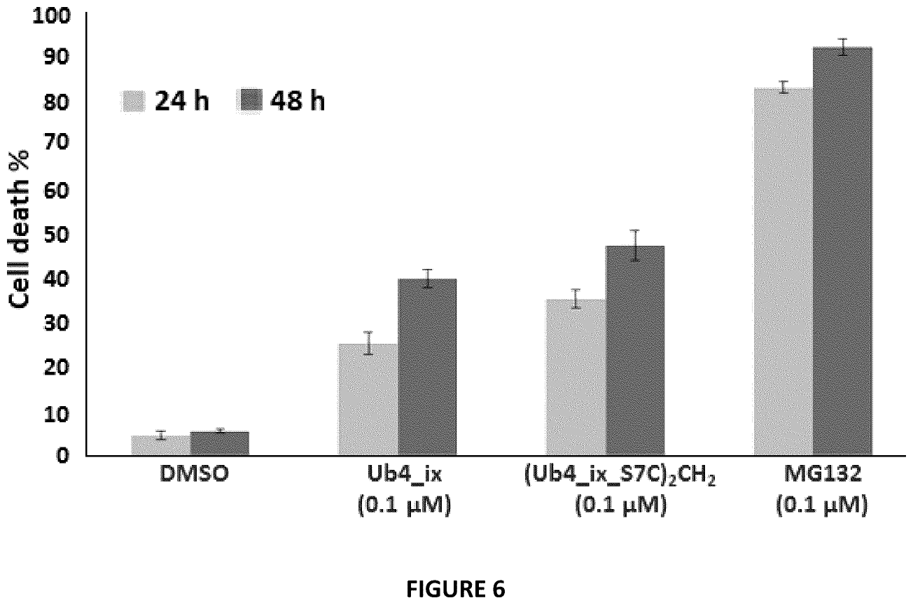

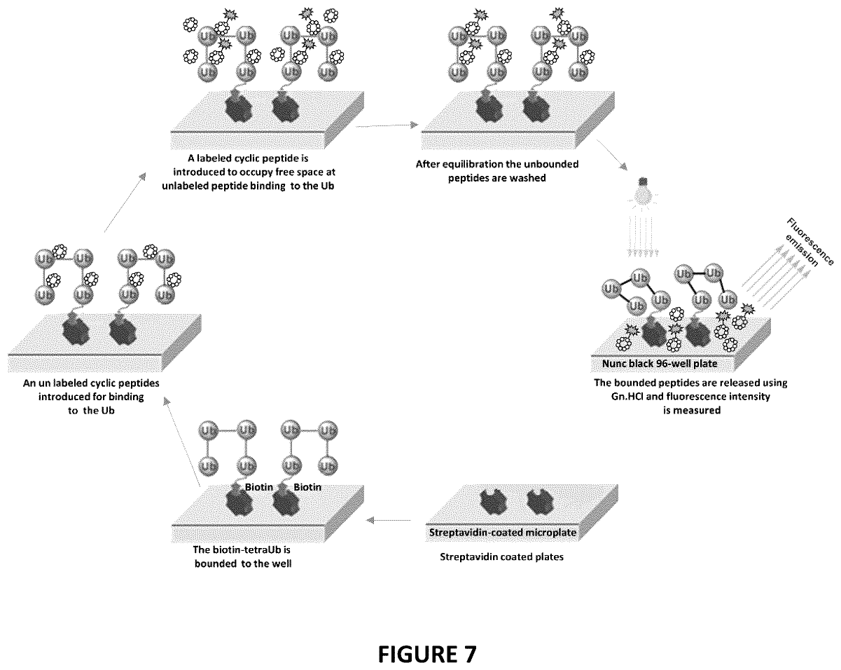

The inventors applied the herein disclosed screening method for the development of peptides with improved binding to tetra-UbK48. For this aim, the inventors used a recently developed cyclic peptide Ub4_ix, which was prepared by SPPS (FIG. 2A), as a scaffold for subsequent manipulations. The inventors first performed an Alanine scan to shed light on the contribution of each residue in sequence and perhaps further modify the sequence to increase its affinity (FIGS. 2 and 6). The inventors found that an analogue Ub4_ix_S7A exhibited slightly improved affinity to tetra-Ub K48 as shown by the ˜13% decrease in the relative fluorescence compared to the core Ub4_ix. After an additional diversification at position “7” in the core scaffold, e.g., Ub4_ix, the inventors detected that each of the mutants Ub4_ix_S7W, Ub4_ix_S7Y, Ub4_ix_S7D (FIG. 2A), and especially Ub4_ix_S7C, 14, (FIG. 2B) further improved the binding parameters (20-40%) (FIG. 3). Notably, Cys is a unique residue with its exceptional nucleophilic character of its thiol side chain, which makes it useful for a late-stage selective modifications.

The inventors took advantage of the observation of incorporating Cys without abolishing the affinity at position “7” for further modifications with a range of small molecules, which could increase the affinity of the peptide. A series of analogues were prepared using selective thiol alkylation, arylation and oxidation chemistries (FIG. 2B). Most of the modified peptides, however, showed unaffected or abolished binding, compared to the initial Ub4_ix scaffold. It has been suggested that bicyclic peptides could possess favorable properties compared to their monocyclic analogues, possibly due to their bivalent binding mode to the target. Accordingly, the inventors decided to test this in the herein disclosed system by preparing a disulfide bond containing dimeric peptide 23, (Ub4_ix_S7C)₂. Indeed, the inventors showed that 23 possess a much greater affinity to the target (˜70% change in the relative signal). Nonetheless, disulfide bond containing peptides may show limited applicability in cellular studies, due to the native reducing environment. To overcome this potential limitation, the inventors designed a dimeric peptide analogue 24, (Ub4_ix_S7C)₂CH₂, bearing a stable thioether-based linker. The obtained stable analogue showed ˜57% change in the relative fluorescence, which is similar to the disulfide containing peptide 23.

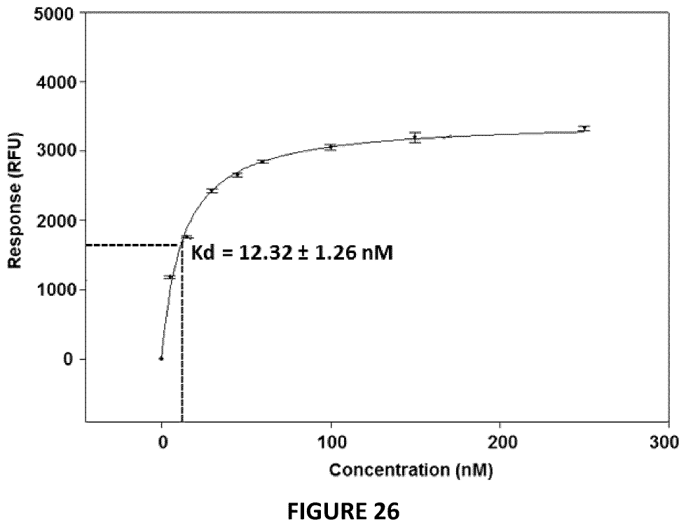

The inventors then applied the herein disclosed modified assay to evaluate the dissociation constants of Ub4_ix and the stapled dimer 24. For this aim, FITC labeled variants of these peptides, Ub4_ix-FITC and (Ub4_ix_S7C)₂CH₂-FITC have been prepared and their K_{d} was determined to be 35.09±2.66 nM and 12.32±1.26 nM, respectively (FIGS. 1G and 26). Notably, K_{d} of Ub4_ix-FITC measured by the herein disclosed assay was in the range of that of Ub4_ix measured by surface plasmon resonance (SPR; 35.09±2.66 vs 6.00±1.00 nM, respectively), which indicates the effectiveness and accuracy of the method.

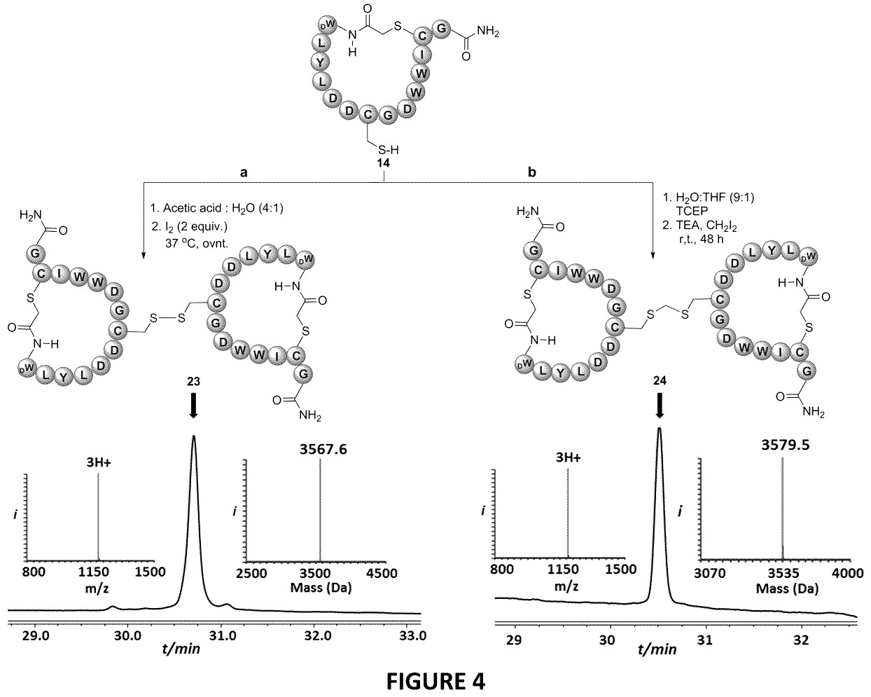

The synthetic route for the preparation of bicyclic analogues and especially of (Ub4_ix_S7C)₂CH₂-FITC labeled stapled dimer was not straightforward and required further attention. To achieve the synthesis of the unlabeled dimers, orthogonally Acm protected Cys7 was incorporated to the scaffold, followed by cleavage from the resin and selective cyclization to give the thioether linked cyclic peptide 13 (FIG. 2B). Subsequently, Acm protecting group was removed by treatment with 10 equiv PdCl₂, followed by quenching the reaction with 40 equiv of dithiothreitol (DTT) at room temperature to give peptide 14, Ub4_ix_S7C, in 42% isolated yield (FIG. 2B). Finally, 14 was dissolved in AcOH:H₂O (4:1) followed by the addition of 2 equiv of 12 at 37° C. for overnight to form 23 in 23% yield (FIG. 4, a). Alternatively, 14 was dissolved in H₂O:THF (9:1) followed by addition of diiodomethane under basic conditions at room temperature for 48 h (FIG. 4, b) to give the 24 in 18% yield.

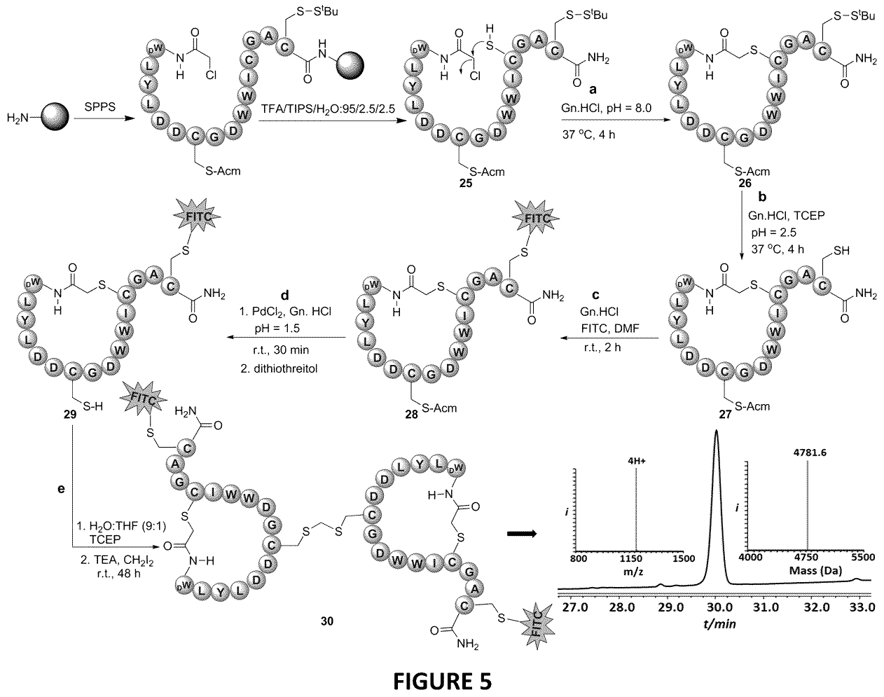

In order to achieve FITC-labeled stapled dimer, both Cys residues had to be orthogonally protected by different protecting groups to enable selective unmasking at a later stage of synthesis. For this aim, the inventors used Acm and tert-butylthiol (—S^{t}Bu) thiol protecting groups. The inventors prepared peptide 25 using SPPS followed by cleavage and selective cyclization to give the thioether linked cyclic peptide 26 (FIG. 5, a). Next, the —S^{t}Bu protecting group was orthogonally removed through selective reduction of a disulfide bond by treatment with tris-(2-carboxyethyl) phosphine (TCEP) in 6 M Gn·HCl pH 2 at 37° C. for 4 h to give 27 (FIG. 5, b) in 42% yield. Subsequently, 2 mM of 27 dissolved in 6 M Gn·HCl (pH 7.5) was treated with 3 equiv of fluorescein-5-maleimide in DMF at room temperature for 2 h to give FITC labeled peptide 28 (FIG. 5, c) in 40% isolated yield. In the following step, the inventors attempted to selectively remove the Acm protecting group using previously developed conditions (Maity et al., 2016). Here, however, under these conditions, the inventors observed disruption of the FITC moiety. To overcome this, the inventors examined a range of different loadings of PdCl₂ at two different temperatures (37° C. and r.t.) under two different pH values (7.5 and 1.5) and for several periods (5, 10, 15, 30 and 45 min). The inventors found that treatment of 28 with 2 equiv of PdCl₂, pH 1.5 at room temperature for 30 min gave the best results for the selective removal of Acm without disruption of FITC, and offered peptide 29 (FIG. 5, d) in 20% isolated yield. Finally a FITC labeled stapled dimer 30, (Ub4_ix_S7C)₂CH₂-FITC, was prepared in 12% yield in a similar fashion as described above (FIG. 5, e).

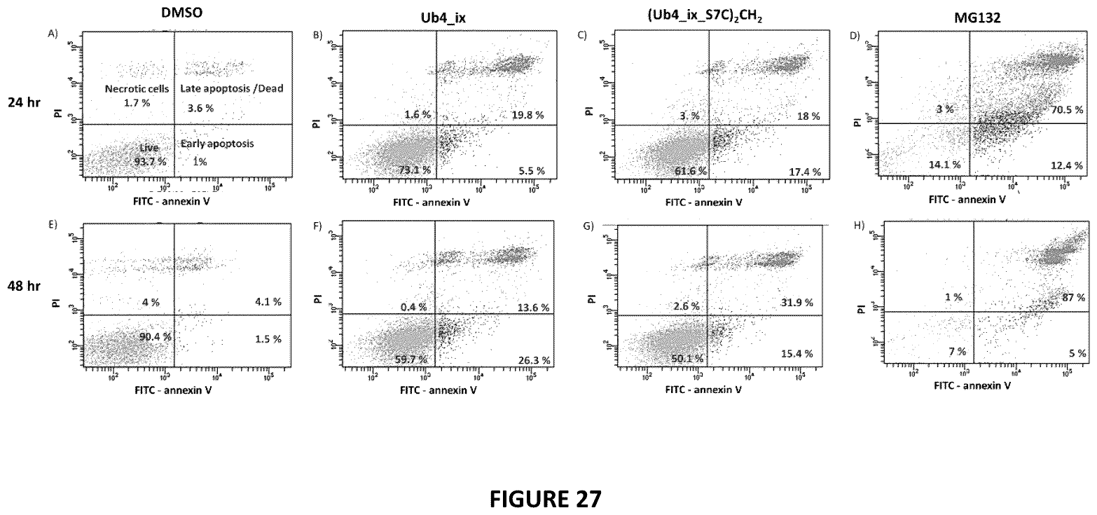

An important and desired property of tetra-Ub targeting peptide is its ability to enter live cells without additional manipulations. The inventors investigated the cellular delivery of 30 (Ub4_ix_S7C)₂CH₂-FITC to HeLa cells using confocal microscopy (FIG. 28). The inventors observed permeability and cytosolic localization of the peptide detected by FITC signal within 2 hours after incubation. Finally, the inventors assessed the cellular effect of (Ub4_ix_S7C)₂CH₂ by conducting viability assay with annexin V-FITC apoptosis/necrosis detection kit (BD Biosciences) using flow cytometry. Treatments with the proteasome inhibitor MG132 and DMSO were performed as positive and negative controls, respectively (FIG. 27). (Ub4_ix_S7C)₂CH₂ was able to induce cell death in Hela cells after 24- and 48-hours incubation in a dose- and time-dependent manner. Furthermore, (Ub4_ix_S7C)₂CH₂ showed increased early- and late stage apoptosis compared to the first-generation peptide Ub4_ix developed by the inventors (FIGS. 6 and 27), which correlates with its improved potency.

To summarize, the inventors have developed a high-throughput, simple and affordable, fluorescence-based competitive assay for screening of peptides against tetra-UbK48. The inventors applied the herein disclosed assay to engineer bicyclic peptides with improved affinity compared to their first-generation analogue. The generation of labeled bicyclic peptides required optimization of stepwise orthogonal deprotection strategy and especially employed modified conditions for selective Cys(Acm) deprotection in the presence of FITC. The herein disclosed bicyclic peptide exhibited cell permeability and induced cancer cell death with greater efficacy, compared to the first-generation monocyclic analogue Ub4_ix. These properties are crucial for a molecule to be considered as a drug candidate. In addition, labeled bicyclic peptide 30 can potentially be used as a polyUbK48-specific staining agent with antibody-like properties, where live cells would be visualized without a need for subsequent permeabilization. This, in turn, will enable exploration of Ub chain dynamics in real-time conditions. Overall, the herein disclosed screening strategy may facilitate the development of modulators of Ub signaling and visualizing agents. A similar principle can be applied for other important and complex targets, including hybrid Ub chains and modified histones.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A cyclic peptide comprising the amino acid sequence WLYLDDCGDWWICG (SEQ ID NO: 13).

2. The cyclic peptide of claim 1, wherein the cysteine residue at position 7 (Cys7) of SEQ ID NO: 13 comprises a chemical modification.

3. The cyclic peptide of claim 2, wherein said chemical modification is selected from the group consisting of: alkylation, arylation and oxidation.

4. The cyclic peptide of claim 1, wherein said peptide comprises not more than 16 amino acids.

5. The cyclic peptide of claim 1, wherein the amino acid at position one of the N terminus is a D amino acid.

6. The cyclic peptide of claim 1, wherein the amino acid at position one of the N terminus is conjugated to a cyclizing molecule.

7. The cyclic peptide of claim 1, being prepared using a cyclizing molecule comprising a halogen.

8. The cyclic peptide of claim 7, wherein said cyclizing molecule is selected from the group consisting of: chloracetyl chloride, 3-chlorobenzoyl (3-ClBz), 4-chlorobenzoyl (4-ClBz) or Cl₂SAc.

9. A dimeric cyclic peptide comprising the cyclic of peptide claim 1.

10. The dimeric cyclic peptide of claim 9, being a homodimer or a heterodimer.

11. The dimeric cyclic peptide of claim 9, comprising two monomeric cyclic peptides covalently linked to one another via cysteine residues.

12. The cyclic peptide of claim 1, being characterized by having: cell penetration capability, ubiquitin (Ub) binding capability, or both.

13. The cyclic peptide of claim 1, being characterized by binding Ub with an affinity Kd ranging from 0.05-100 nM.

14. A pharmaceutical composition comprising the cyclic peptide of claim 1,
and an acceptable carrier.

15. The pharmaceutical composition of claim 14, characterized by having pro-apoptotic activity.

16. (canceled)

17. A method for treating a subject afflicted with cancer, comprising administering to said subject a therapeutically effective amount of the pharmaceutical composition of claim 14,
thereby treating the subject afflicted with cancer.

18. The method of claim 17, wherein said treating comprises reducing deubiquitination activity in at least one cell of said subject.

19. The method of claim 17, wherein said treating comprises reducing ubiquitinated proteins proteasomal degradation rate in at least one cell of said subject.

20.-34. (canceled)
